SEQUENCE LISTING (30 sequences)

SEQ ID NO: 1 - protein, 5 aa, Artificial sequence
  synthetic peptide
  MOD_RES (1)..(1): amidation by a Palmitoyl chain on the N terminal end
KTTKS

SEQ ID NO: 2 - protein, 4 aa, Artificial sequence
  synthetic peptide
  MOD_RES (1)..(1): amidation by a Palmitoyl chain on the N terminal end
GQPR

SEQ ID NO: 3 - protein, 6 aa, Artificial sequence
  synthetic peptide
  MOD_RES (1)..(1): amidation by a Palmitoyl chain on the N terminal end
VGVAPG

SEQ ID NO: 4 - protein, 5 aa, Artificial sequence
  synthetic peptide
  MISC_FEATURE (1)..(1): Xaa being a lysine, hydroxylysine, ornithine, ldiaminobutyric acid,diaminopropionic acid or formyl, acetyl, trifluoroacetyl, methanesulfonyle or succinyl derrivatives
  MOD_RES (1)..(1): X = -CO-R1 or -SO2-R1 or biot group, R1 = C1- C24 group
  MISC_FEATURE (4)..(4): Xaa being a lysine, hydroxylysine, ornithine, ldiaminobutyric acid,diaminopropionic acid or formyl, acetyl, trifluoroacetyl, methanesulfonyle or succinyl derrivatives
  MISC_FEATURE (5)..(5): Z = -OR1, -NH2, -NHR1 ou -NR1R2 ; R1, R2 = C1- C24 group
  MISC_FEATURE (5)..(5): Xaa being a serine or a threonin
XTSXX

SEQ ID NO: 5 - protein, 5 aa, Artificial sequence
  synthetic peptide
  MOD_RES (1)..(1): amidation by a Palmitoyl chain on the N terminal end
KTSKS

SEQ ID NO: 6 - protein, 6 aa, Artificial sequence
  synthetic peptide
  MOD_RES (1)..(1): amidation by a Palmitoyl chain on the N terminal end
GKTTKS

SEQ ID NO: 7 - protein, 5 aa, Artificial sequence
  synthetic peptide
  MOD_RES (1)..(1)
  MOD_RES (1)..(1): amidation by a Palmitoyl chain on the N terminal end
KTSKT

SEQ ID NO: 8 - protein, 6 aa, Artificial sequence
  synthetic peptide
  MOD_RES (1)..(1): amidation by a Palmitoyl chain on the N terminal end
KTSKSA

SEQ ID NO: 9 - protein, 4 aa, Artificial sequence
  Synthetic peptide
  MOD_RES (1)..(1): amidation by a Palmitoyl chain on the N terminal end
KTFK

SEQ ID NO: 10 - protein, 4 aa, Artificial sequence
  synthetic peptide
GQPR

SEQ ID NO: 11 - protein, 4 aa, Artificial sequence
  synthetic peptide
RSRK

SEQ ID NO: 12 - protein, 4 aa, artificial sequence
  peptidic compound
KTFK

SEQ ID NO: 13 - protein, 4 aa, Artificial sequence
  Synthetic peptide
KTAK

SEQ ID NO: 14 - protein, 4 aa, Artificial sequence
  Synthetic peptide
KAYK

SEQ ID NO: 15 - protein, 4 aa, Artificial sequence
  Synthetic peptide
KFYK

SEQ ID NO: 16 - protein, 4 aa, Artificial sequence
  synthetic peptide
TKPR

SEQ ID NO: 17 - protein, 5 aa, Artificial sequence
  synthetic peptide
KTTKS

SEQ ID NO: 18 - protein, 6 aa, Artificial sequence
  synthetic peptide
GKTTKS

SEQ ID NO: 19 - protein, 6 aa, Artificial sequence
  synthetic peptide
VGVAPG

SEQ ID NO: 20 - protein, 4 aa, Artificial sequence
  Synthetic peptide
  MOD_RES (1)..(1): amidation by a Elaidoyl chain on the N terminal end
KTFK

SEQ ID NO: 21 - protein, 4 aa, Artificial sequence
  Synthetic peptide
  MOD_RES (1)..(1): amidation by a Elaidoyl chain on the N terminal end
KTAK

SEQ ID NO: 22 - protein, 4 aa, Artificial sequence
  Synthetic peptide
  MOD_RES (1)..(1): amidation by a Elaidoyl chain on the N terminal end
KAYK

SEQ ID NO: 23 - protein, 4 aa, Artificial sequence
  Synthetic peptide
  MOD_RES (1)..(1): amidation by a Elaidoyl chain on the N terminal end
KFYK

SEQ ID NO: 24 - protein, 5 aa, Artificial sequence
  synthetic peptide
  MOD_RES (1)..(1): amidation by a Palmitoyl chain on the N terminal end
  MISC_FEATURE (5)..(5): Xaa being either a Proline P or a Leucine L.
YGGFX

SEQ ID NO: 25 - protein, 6 aa, Artificial sequence
  Synthetic peptide
  MOD_RES (1)..(1): amidation by a Palmitoyl chain on the N terminal end
  MISC_FEATURE (6)..(6): Xaa being Trp, Phe, Tyr, Tic, 7-hydroxy-Tic or Tpi
HLDIIX

SEQ ID NO: 26 - protein, 6 aa, Artificial sequence
  synthetic peptide
  MOD_RES (1)..(1): Acetylation on the N-terminal end
EEMQRR

SEQ ID NO: 27 - protein, 5 aa, Artificial sequence
  synthetic peptide
YAGFL

SEQ ID NO: 28 - protein, 4 aa, Artificial sequence
  synthetic peptide
AHSH

SEQ ID NO: 29 - protein, 6 aa, Artificial sequence
  synthetic peptide
SIKVAV

SEQ ID NO: 30 - protein, 6 aa, Artificial sequence
  synthetic peptide
GPQGPQ

(19) United States
(12) Patent Application Publication
(10) Pub. No.: US 2023/0255873 A1
(43) Pub. Date: Aug. 17, 2023

(54) COSMETIC OR DERMATOLOGICAL PEPTIDE-BASED TREATMENT OF THE SKIN AND ITS INTEGUMENTS

(71) Applicant: Sederma, Le Perray-en-Yvelines (FR)

(72) Inventors: Philippe Mondon, Montrouge (FR); Caroline Ringenbach, Rambouillet (FR); Olga Gracioso, Courbevoie (FR)

(73) Assignee: Sederma, Le Perray-en-Yvelines (FR)

(21) Appl. No.: 18/018,412

(22) PCT Filed: Jul. 28, 2021
(86) PCT No.: PCT/EP2021/071120

§ 371 Date: Jan. 27, 2023

(30) Foreign Application Priority Data
Jul. 30, 2020 (FR) FR2008081

(51) Int. Cl. A61K 8/64; A61Q 19/08; A61Q 17/00; A61K 38/08; A61P 17/02

(52) U.S. Cl. CPC A61K 8/64; A61Q 19/08; A61Q 17/005; A61K 38/08; A61P 17/02

(57) ABSTRACT

The treatment according to the invention provides for the use of at least one peptide of general Formula X-(Xaa)ₙK*TSK*X′aa-(Xaa)ₘZ for a non-therapeutic cosmetic treatment of keratin materials of the skin and of its integuments, including treatment of the epidermis, scalp, hair, and nails, where X, Xaa, K*, X′aa, Z, m and n are as defined. A preferred peptide is Pal-KTSKS.

### TECHNICAL FIELD

The present invention relates to a cosmetic or dermatological peptide-based treatment of the skin and its integuments, of human mammals or animals. The invention relates more particularly to cosmetic, dermatological, and hygiene and personal care products industries.

### BACKGROUND ART

Peptides have an important signal function and coordinate many biochemical processes. Therefore, they have for many years become essential and promising active ingredients, more particularly in the cosmetics industry where new compounds able to embellish the skin and integuments, namely, to improve their general condition, are searched.

Most of the peptides currently available are peptides that act on the dermis by stimulating components of the extracellular matrix, primarily collagen and elastin. Numerous peptides are proposed in this area, in particular by the Applicant, such as the Pal-KTTKS (SEQ ID No 1) sold under the Matrixyl® trademark, the Pal-GHK and Pal-GQPR mixture (SEQ ID No 2) sold under the Matrixyl® 3000 trademark, the Pal-KMO₂K sold under the Matrixyl®synthe′6® trademark (MO₂ corresponding to a dioxygenated methionine) or more recently Pal-K(P)HG (with a proline grafted on a lysine) sold under the Matrixyl®Morphomics® trademark, or alternatively the Pal-VGVAPG (SEQ ID No 3) sold under the Dermaxyl™ or Biopeptide EL™ trademarks and the N-acetyl-Tyr-Arg-O-hexadecyl ester sold under the Idealift™ or Calmosensine™ trademarks.

However, skin beauty and good health also depend to a large extent on the quality and thickness of the epidermis, in particular via an optimal differentiation of the keratinocytes, and on the epidermis capacity to form its outermost layer, the horny layer or stratum corneum, and to renew this layer regularly by desquamation. The epidermis, and in particular its horny layer, in fact forms a real skin barrier essential to protect itself from molecules and attacks from the external environment (light radiation, pollutants, bacteria, viruses, allergens, plant toxins, etc.). Thanks to a good quality epidermis and effective protection by this skin barrier, the risks of microinflammations of the epidermis is for example limited, which can cause premature skin aging, a protection which is particularly necessary for sensitive skin. The risk of water loss is also limited, which helps to maintain good hydration of the epidermis. In addition to this physical barrier, there is also a chemical barrier formed by antimicrobial peptides synthesized by keratinocytes, the role of which bieng to protect the skin from pathogenic bacteria. It is also important to preserve and improve this chemical barrier.

In addition, the skin microbiota, a very complex ecosystem made up of a set of living microorganisms (bacteria, yeasts, viruses, and parasites), has several functions: role of defense, skin barrier, and regulator of the immune system. It is important to protect its balance by preventing, for example, that certain species by developing excessively do not cause damage to the skin. This is the case, for example, with yeasts of the Malassezia genus, involved in dandruff conditions, as well as Propionibacterium acnes (recently renamed Cutibacterium acnes), the acne responsible bacteria. The latter, although part of the normal micro-flora of the skin, by multiplying too quickly, will promote the proliferation and migration of keratinocytes, participate in the formation of radical species such as superoxide anion and cause a cascade of reactions which results in the production of pro-inflammatory molecules and thus contribute to the development of acne.

Yeasts of the Malassezia genus are part of the normal micro-flora of the scalp. When they can multiply quickly enough, they cause dandruff conditions.

It is therefore important to preserve the balance of the skin microbiota, by controlling the excessive multiplication of certain bacteria causing skin disorders.

### SUMMARY OF THE INVENTION

The object of the present invention is to provide a peptide which can be used in cosmetics and dermatology which meets these needs.

To this end, according to a first object the present invention proposes the use of at least one peptide of the following general Formula 1:

X—(Xaa)ₙK*TSK*X′aa-(Xaa)ₘ-Z

for a non-therapeutic cosmetic treatment of the keratinic materials of the skin and its integuments (comprising eyelashes and eyebrows), comprising the treatment of epidermis, scalp, hair, and nails, wherein in general Formula 1:
K* is chosen from lysine, hydroxylysine, ornithine, diaminobutyric acid or diaminopropionic acid or their formyl, acetyl, trifluoroacetyl, methanesulfonyl or succinyl derivatives, the two K* possibly being identical or different;
(Xaa)ₙ and (Xaa)ₘ correspond independently of one another to a sequence of n or m amino acids Xaa chosen independently of one another from G, A, P, V, L, I and F, with n and m being integers which may be equal or different between 0 and 5;
X′aa is chosen from threonine and serine;
at the N-terminal end X is chosen from H, —CO—R¹, —SO₂—R¹ or a biotinoyl group;
at the C-terminal end Z is chosen from OH, OR1, NH², NHR¹ or NR¹R²; and
R¹ and R² are, independently of one another, chosen from an alkyl, aryl, aralkyl, alkylaryl, alkoxy, saccharide and aryloxy group, which may be linear, branched, cyclic, polycyclic, unsaturated, hydroxylated, carbonylated, phosphorylated and/or sulfurized, said group having from 1 to 24 carbon atoms and possibly having in its backbone one or more O, S and/or N heteroatoms.

This treatment is purely cosmetic. It differs from a therapeutic treatment insofar as it is aimed at skin and its integuments in a healthy state (as opposed to a pathological state), for beautifying it or avoiding disorders (as a preventive measure), in particular aesthetic or sensory disorders.

In vitro test results on keratinocyte culture (in particular on DNA-Microarray) and on the proliferation of microorganisms in culture are given below in the description and show a biological action of the peptide according to invention at several levels, in particular:
Stimulation of the production of several molecules constituting the cutaneous barrier or intervening positively in the keratinocyte differentiation at the origin of this protective physical barrier (the horny layer or stratum corneum), the peptide acting at all levels of the barrier formation process: from the filaggrin metabolization to the stratum corneum element production, the formation of lipidic lamellar bodies, and the formation of tight junctions.
The decrease in the level of a certain number of molecules involved in skin microinflammations caused by the multiple small attacks of day life (light radiation, pollutants, etc.) and by certain behaviors (cigarettes, sugary or too fatty foods, etc.). The peptide according to the invention can strengthen the skin's defense system against bacteria, oxidants, and radiation by slowing down or preventing the micro-inflammations resulting from these toxic agents. This beneficial effect is manifested by the overexpression of various genes, including in particular those encoding the cytokine IL-37, human beta-defensin 3, annexin A1 and Serine protease-8 in the DNA-Microarray. It can also be seen in the effect of inhibiting the production of pro-inflammatory mediators (PGE2 and IL-6) by keratinocytes that have been exposed to UVB irradiation.
The DNA-Microarray shows that many of the proteins involved in the formation of the natural hydration factor are overexpressed in the presence of the peptide according to the invention, in particular filaggrin and the enzymes involved in its degradation. The moisturizing aspect is reinforced by a test which shows a dose-dependent increase in the synthesis of hyaluronic acid, in the presence of the peptide according to the invention, on keratinocytes in culture. By capturing water, the hyaluronic acid molecule expands and becomes perfectly resistant to compression, thus giving the epidermis elastic properties, ensuring good hydration, and contributing to a smoother skin.
The DNA-Microarray also shows that the kallikreins are overexpressed in the presence of the peptide according to the invention. These are proteases involved in the renewal of the horny layer allowing natural desquamation and thus ensuring a soft “natural” smoothing effect. The increased expression and synthesis of these enzymes (seen with the KLK5 marker in the DNA-Microarray) improves the natural process of corneocyte desquamation and is similar to a natural peel. The peptide according to the invention is thus suitable for smoothing the relief of the skin, in particular epidermal scars such as residual traces of acne lesions (such as atrophic scars).
The strong inhibitory effect of the peptide is shown on a growth curve of the acne bacteria Propionibacterium acnes (now re-named Cutibacterium acnes). Furthermore, the DNA-Microarray shows that the invention peptide strongly stimulates the expression of many anti-microbial peptides (AMPs), which are considered to be a chemical skin barrier because they protect the skin against pathogenic microorganisms of the environment. These peptides are human beta defensin 1 and 3, RNase 7, elafin, and psoriasin. They exhibit a broad spectrum of destruction against gram+ and gram− bacteria and yeasts.
The anti-microbial peptides, strongly stimulated by the invention peptide, also act by modulating the innate immune responses, to obtain protection against infection and control of inflammation and healing, and in order to initiate adaptive immune responses. Human beta-defensin-3 (HBD3), for example, is a key molecule in the cutaneous immune system, a marker of the immune barrier function of the skin.
Detoxifying action: DNA-Microarray shows a strong stimulation of Heme Oxygenase 1, an antioxidant enzyme, cytoprotective against the effects of oxidative stress.
The DNA-Microarray shows that a certain number of compounds of the hair itself and of compounds involved in its formation or maintenance have their gene expression increased following contact with the invention peptide. This is the case with trichohyaline, corneodesmosine, skin aspartic protease, arachidonate 12-lipoxygenase, transglutaminase 3 and periplakin.
The DNA-Microarray shows the geneoverexpression of encoding proteins that make up the nail or are involved in its formation (Trichohyaline, Filaggrin and Periplakin).

These results show that the cosmetic treatment according to the invention is indeed suitable for protecting the epidermis and the scalp from external aggressions liable to cause damages, such as microorganisms, radiation, and molecules, by treating the stratum corneum, thanks in particular to the preservation or improvement of this skin barrier (physical and chemical) of the epidermis. More specifically, thanks to these characteristics, the cosmetic treatment is suitable for:
Protecting (preventing) the epidermis against redness, irritation, and tightness, and/or protect the epidermis against premature aging; and/or
Protecting the scalp against the appearance of dandruff (preventive action), in particular by slowing down or inhibiting the development of the yeasts responsible for the dandruff state (of the Malassezia genus); and/or
Protecting the epidermis against acne (preventive action), in particular by slowing down or inhibiting the multiplication of the bacteria responsible for acne (Propionibacterium acnes).

The tests have also shown that the cosmetic treatment according to the invention is adapted for:
Preserving and strengthening the hydration of the epidermis, in particular its upper part, and/or
Smoothing the epidermis, in particular smoothing traces of acne already formed (residual) and unsightly, and/or
Protecting the skin microbiota (its balance) and strengthen skin immunity, and/or
A detoxifying action, and/or
For uniformizing skin epidermis, and/or
For treating skin pores, and/or
A beneficial action on the nails (maintenance of healthy nails or treatment of damaged nails), and/or
A beneficial on the hair (scalp and body including eyelashes and eyebrows), in particular on their vigor and the growth.

These results thus show in particular that the use of the peptide according to the invention is particularly advantageous for treating oily and/or acne-prone skins via a preventive action. This type of skin often corresponds in adolescence to oily skin due to excess sebum for hormonal reasons. The Propionibacterium acnes bacteria develops in the hair follicle sheath anaerobically by feeding on sebum and producing in turn waste, in particular dead cells. By preventing or limiting the proliferation of the bacteria, the developpement of waste in the hair follicle sheath is then first prevented, waste that would otherwise have led to an obstruction of the hair follicle sheath (forming black dots), to inflammations and finally to acne pimples whose treatment falls thereafter within dermatology.

The reduction in the amount of Propionibacterium acnes therefore makes it possible to prevent the progression toward a pro-acne state, in particular a pro-inflammatory state.

Thus, the peptide of the invention, in a purely cosmetic point of view, can limit the emergence of conditions conducive to acne, especially those producing micro-comedones: the production of sebum is reduced; the epidermal barrier function and the maturation conditions of keratinocytes to limit their hyper-proliferation are improved; the explosive growth of P. acnes and the formation of its biofilm, control conditions leading to the formation pro-inflammatory molecules are limited. The petide can also act downstream, on the appearance of the scars left by acne episodes by smoothing the skin surface to give it a more aesthetic appearance.

Also, from a non-therapeutic cosmetic point of view, the results show that the use of the peptide according to the invention is particularly suitable and advantageous for treating (curative effect) an unsightly skin caused by scars or traces of acne remaining after an acne attack, the treatment comprising or consisting of an epidermis smoothing treatment.

According to another object, the present invention provides the use of a peptide of the following general Formula 1:

X—(Xaa)ₙK*TSK*X′aa-(Xaa)ₘ-Z

wherein in general Formula 1:
K* is chosen from lysine, hydroxylysine, ornithine, diaminobutyric acid or diaminopropionic acid or their formyl, acetyl, trifluoroacetyl, methanesulfonyl or succinyl derivatives, the two K* possibly being identical or different;
(Xaa)ₙ and (Xaa)ₘ correspond independently of one another to a sequence of n or m amino acids Xaa chosen independently of one another from G, A, P, V, L, I and F, with n and m being integers which may be equal or different between 0 and 5;
X′aa is chosen from threonine and serine;
at the N-terminal end X is chosen from H, —CO—R¹, —SO₂—R¹ or a biotinoyl group;
at the C-terminal end Z is chosen from OH, OR¹, NH₂, NHR¹ or NR¹R²; and
R¹ and R² are, independently of one another, chosen from an alkyl, aryl, aralkyl, alkylaryl, alkoxy, saccharide and aryloxy group, which may be linear, branched, cyclic, polycyclic, unsaturated, hydroxylated, carbonylated, phosphorylated and/or sulfurized, said group having from 1 to 24 carbon atoms and possibly having in its backbone one or more O, S and/or N heteroatoms, for the preparation of a cosmetic or dermatological composition, said composition comprising the peptide in a physiologically acceptable medium.

In particular, in the case of a dermatological composition, the latter will suitable for an antimicrobial (antibacterial and/or antifungal) and/or anti-inflammatory curative treatment, this, as seen above, thanks to the inhibitory effect of the peptide shown on a growth curve of the acne bacterium Propionibacterium acnes and thanks to the strong stimulation of the expression of a large number of anti-microbial peptides (AMPs), in particular capable of inhibiting the growth of yeasts of the Malassezia genus responsible for dandruff, thanks to the repair of the cutaneous defense system (anti-inflammatory and immune) against bacteria, oxidants, radiation, thanks to the reconstitution of the cutaneous barrier and thanks to the reinforcement of hydration.

The present invention therefore also provides the peptide for a therapeutic treatment comprising the application to a skin in need thereof of an effective amount of the peptide according to the invention, the treatment being in particular antimicrobial (antibacterial or antifungal), and/or anti-inflammatory, said treatment being in particular suitable for soothing sensitive and irritated skin and/or for treating acne, psoriasis, dermatitis and eczema.

According to the invention, the peptide is suitable for use in the preparation of a cosmetic composition for treating acne-prone skin and/or for hydrating and/or smoothing skin and/or for preventing the appearance of dandruff and/or for protecting the skin microbiota and/or strengthening skin immunity.

The peptide used according to the invention is characterized in that it contains at least the amino acid sequence K*TSK*X′aa, X′aa being T or S, a sequence which is biologically active on keratinocytes. Sequences of 1 to 5 non-polar amino acids chosen from G, A, P, V, L, I and F, can be added on either side of the active sequence K*TSK* X′aa, preferably chosen from G, A and F, more preferably G and A.

Preferably, according to the invention K* is a lysine K or an ornithine, more preferably a lysine. More preferably according to the invention n and m are independently of one another equal to 0, 1 or 2, preferably are equal to 0, the peptide having the general Formula 2: X—K*TSK*X′aa−Z (SEQ ID No 4).

Preferably, the peptide according to the invention has the general Formula 3: X-KTSKX′aa-Z, X and Z and X′aa being as defined above.

Preferably, the peptide according to the invention is modified in the N-terminal position and/or in the C-terminal position, preferably either in the N-terminal position or in the C-terminal position, preferably in the N-terminal position only.

According to other prefered features of the invention:
R¹ and/or R² is an alkyl chain of 1 to 24 carbon atoms, preferably a lipophilic alkyl chain of 3 to 24 carbon atoms; and/or
X is an acyle groupe CO—R¹; preferably selected from an octanoyl (C₈), decanoyl (C₁₀), lauroyl (C₁₂), myristoyl (C₁₄), palmitoyl (C₁₆), stearoyl (C₁₈), biotinoyl, elaidoyl, oleoyl and lipoyl; more preferably selected from lauroyl (C₁₂), myristoyl (C₁₄) and palmitoyl (C₁₆), and/or
Z is selected from OH, OMe, OEt and NH₂, preferably OH; and/or
X is selected from palmitoyl (C₁₆), myristoyl (C₁₄) and lauroyl (C₁₂); more preferably X is a palmitoyl (C₁₆) and Z is OH; and/or
X′aa is the Serine (S).

Peptides comprising in the N or C terminal position derivatives of particular acids such as those of ascorbic, retinoic, cinnamic, oleanolic, hyaluronic, nicotinic, lipoic, gallic or pantothenic acid are also within the scope of the present invention.

A preferred peptide according to the invention is thus the X-KTSKS-Z, more preferably the Pal-KTSKS-OH (also called Pal-KTSKS, SEQ ID No 5), corresponding to a substitution with a palmitoyl chain on the N-terminal side (X=Pal) and no substitution on the C-terminal side (Z=OH).

Another example of a preferred peptide is the Pal-GKTSKS (SEQ ID No 6).

The peptide according to the invention can be optically pure or can consist of the L or D isomers or a mixture thereof. L isomers which are those naturally occurring may be preferred.

The peptide can be obtained in particular by a synthetic or biotechnological route.

The peptide can optionally be in the form of a salt, in particular a hydrochloride or acetate.

The present invention also covers:
derivatives of the peptide (with modification and/or addition of a chemical function but without change in the carbon skeleton),
analogs (with modification and/or addition of a chemical function but in addition with a change in the carbon skeleton), and
complexes with other species such as a metal ion (eg copper, zinc, manganese, magnesium).

For its use according to the invention, the peptide can be dissolved in a physiologically acceptable lipophilic or hydrophilic matrix, optionally with a solubilizer, depending on the dosage form envisaged. The term “composition according to the invention” means either a composition constituting an active ingredient intended to be formulated, or a composition for an end consumer.

According to the present invention, the term “physiologically acceptable medium” means, without being limiting, an aqueous or aqueous-alcoholic solution, an alcoholic solution, a glycolic or hydroglycolic solution, a water-in-oil emulsion, an oil-in-water emulsion, a microemulsion, an aqueous gel, an anhydrous gel, a serum, a vesicle dispersion, or a powder.

“Physiologically acceptable” means that the medium is suitable for a topical or transdermal use, in contact with mucous membranes, nails, scalp, hair, mammalian and more particularly human hair and skin, the composition capable of being ingested or injected into the skin, without risk of toxicity, incompatibility, instability, allergic response, and the like. This “physiologically acceptable medium” forms what is conventionally called the excipient of the composition.

The peptide according to the invention can be used in a vectorized form, in a bound form, incorporated or adsorbed on/to macro-, micro-, and nanoparticles, as capsules, spheres, liposomes, oleosomes, chylomicrons, sponges, microemulsions or nanoemulsions form, or adsorbed, for example, on powdery organic polymers, talc, bentonites, spores or exines and other mineral or organic supports.

A composition comprising the peptide according to the invention can be provided in any galenic form (examples are given below) and also be vehiculed via a textile support made of natural or synthetic fibres, wool, or any material suitable for coming into contact with the skin, or which can be used in clothing, such as day or night underwear, handkerchiefs, or tissues, in order to permits cosmetic or dermatological effect through this skin/textile contact and allow topical delivery. Particularly and advantageously, according to the invention, the peptide can be associated to at least one additional active adapted to reinforce the activity and/or to act in a complementary manner on one or more other activities. These additional active agents can be incorporated at the stage of the ingredient intended for the formulator, or else at the final stage in the composition forming the cosmetic product intended for the consumer.

Various additional active ingredients for this purpose are mentioned below in the detailed description.

According to the invention, a method is also provided for improving the aesthetic appearance of the skin and its integuments comprising the topical application to the skin of an effective amount of a cosmetic composition comprising at least one peptide according to the invention as described above.

«Topical treatment» or «topical use» means according to the invention, an application that is intended to act where it is applied: skin, mucosa and/or integuments.

The composition according to the invention may be applied locally to targeted areas.

The «effective» amount depends on various factors, such as the age, the condition of the skin and integuments of the person, seriousness of the disorder(s) or pathology, the administration mode, etc. An effective amount means a non-toxic amount enough to achieve the desired effect, more or less pronounced.

In a cosmetic composition according to the invention, the at least one peptide, in order to be present in an effective amount, is generally present in proportions of between 0.1 ppm and 1000 ppm relative to the total weight of the composition, preferably between 0.5 ppm and 200 ppm, more preferably between approximately 1 ppm and 100 ppm, depending on the destination of the composition and the desired effect more or less pronounced.

In a dermatological composition according to the invention, the at least one peptide to be present in an effective amount, is generally found in greater proportions than in cosmetics.

All percentages and ratios used herein are by weight of the total composition and all measurements are made at 25° C. unless it is otherwise specified.

For example, for a cosmetic treatment of the face, the European Cosmetics Directive has set a standard amount for applying a cream of 2.72 mg/cm²/day/person and for a body lotion of 0.5 mg/cm²/day/person.

According to other specific features, the cosmetic treatment method according to the invention can be combined with one or more other treatment methods targeting the skin such as lumino-therapy, heat, vibration, electroporation, micro-needle patch or aromatherapy treatments.

According to the invention, devices with several compartments or kits may be proposed to apply the method described above which may include for example and non-restrictively, a first compartment containing a composition comprising the at least peptide of the invention, and in a second compartment an excipient and/or an additional active ingredient and/, the compositions contained in the said first and second compartments in this case being considered to be a combination composition for a simultaneous, separate or stepwise use in time, particularly in one of the treatment methods recited above.

A composition according to the invention is also suitable for a therapeutic treatment, in particular a skin treatment, in particular also of a skin having a diseased epidermis, at suitable doses.

### DETAILED DESCRIPTION

The present invention will be better understood in the light of the following description of an embodiment and of in vitro tests.

A—Example of Preparation of the Pal-KTSKS (SEQ ID No 5) Peptide According to the Invention

The Pal-KTSKS peptide is prepared by peptide synthesis. A serine is coupled with a resin via its terminal acid function (with a coupling agent, for example DCC (diclyclohexylcarbodiimide)/NHS (N-hydroxysuccinimide) or HBTU (2-(1H-benzotriazol-1-yl) -1, 1, 3, 3-tetramethyluronium hexafluorophosphate)/HOBT (1-hydroxy-benzotriazole)). The serine thus protected is then reacted with a lysine derivative in the presence of a coupling agent, then the same operation is carried out to add a second serine and a threonine, then in the same way to add the second lysine. The latter is then acylated on its amine function with an activated palmitic acid derivative (palmitoyl chloride for example) in the presence of a base. The peptide chain is cleaved from the resin in an acidic medium and after precipitation, washing and drying, the product palmitoyl-lysyl-threonyl-serine-lysyl-serine is obtained in solid form.

The peptide according to the invention can also be prepared by a biotechnological route, via a microorganism capable of producing it at least partially.

B—Preparation Example of a Cosmetic Active Ingredient According to the Invention Comprising the Pal-KTSKS (SEQ ID No 5)

The Pal-KTSKS peptide is amphiphilic, the Pal chain being hydrophobic and the peptide part being hydrophilic. The peptide, for example at 1200 ppm, is dissolved in a water/glycol matrix (the medium) with suitable surfactants. The active ingredient can be prepared in a concentration range of 100 to 10,000 ppm of the peptide(s) of the invention.

C—In-Vitro Efficacy Tests

I—on the Preferred Peptide of the Invention: The Pal-KTSKS

1—Description of Performed Tests

They were carried out on epidermis cells: normal human keratinocytes (NHK) or human keratinocytes (HaCaT), and dermis cells: normal human fibroblasts (NHF). A test was carried out on a culture of the Propionibacterium acnes bacterium. The peptide was tested in solution in an inert solvent at concentrations recommended for a cutaneous use.

1.1—Immuno-Enzymatic Assays (ELISA) on Normal Human Keratinocyte (NHK) Culture

Principle: NHKs in culture and at confluence are brought into contact with the peptide of the invention at different concentrations for 24 hours in a medium allowing their survival. Then the cell layers are irradiated with UVB (light stress, intended to mimic an experimental cutaneous micro-inflammation) in a physiological buffer and again brought into contact with the products to be tested for 24 hours. At the end of this incubation, the culture media are assayed by ELISA to evaluate the amounts of pro-inflammatory mediators (PGE2 and IL-6) produced by these cells in response to irradiation. The results are compared to the control. A decrease in the amount of mediators produced will be interpreted as a limitation of damages related to inflammation. An estimate of the cell number is made on the attached layer to reduce the assay results to the number of cells. A study of variances and a Student t test are carried out to judge the significance of the results.

This same type of test was carried out on a HaCaT culture (human keratinocyte line), but without the irradiation step, with a contact time of 72 hours between the cells and the peptide of the invention and an ELISA assay of hyaluronic acid on the culture medium.

1.2—DNA Microarray-Technology NHK Culture

Principle: the peptide according to the invention at 9 ppm is brought into contact for 24 hours with confluent NHKs (versus control case). Then the NHKs layers are rinsed, and the cells are crushed to extract their mRNA. These mRNAs are then converted into DNA sequences which are analyzed after deposition on DNA chips and amplification by a method similar to qRT-PCR (Real-time Quantitative Reverse Transcription Polymerase Chain Reaction). The mRNA variations due to the peptide are compared to the control case (the solvent of the peptide). The results are expressed as an expression ratio between the treated case and the control case. A ratio greater than 2 is considered to be induction of gene expression (+50% compared to the control).

1.3—Immuno-Enzymatic Tests on Normal Human Fibroblasts (NHF) Culture

Principle: Normal Human Fibroblasts (NHF) are cultured in 24-well plates for 24 hours in the proliferation medium until confluence. The cells are then brought into contact or not (control case) with the preferred peptide according to the invention for 3 days in a medium without serum. The culture media are collected and the concentrations of various elements of the dermis or the basement membrane (PIP (carboxy-terminal propeptide of procollagen type I), collagen I, collagen IV, fibronectin) are measured by ELISA assays. An estimate of cell viability is also carried out at the end of culture, in order to weight the data from the ELISA, using an assay with the Hoechst 33258 reagent (DNA intercalator). A study of variances and a Student t test for paired series were carried out to judge the significance of the results.

1.4—Propionibacterium acnes Growth Inhibition Test

Principle: Suspensions of Propionibacterium acnes of equivalent density are cultured in a suitable medium in the presence (test case) or absence (control case) of the preferred peptide according to the invention, in anaerobic condition, at 37° C. Samples are taken at regular intervals to measure the OD at 600 nm, to follow the growth of the bacterial population over time. The growth curve of each culture can therefore be established.

1.5—Scratch Test on HaCaTs

Principle: HaCats are inoculated in a 6-well plate in a suitable medium until confluence. 24-hour contact with the peptide according to the invention (test cases) or its excipient (control case) in the culture medium is then carried out. After this first step, wounds are made on the cell layers in the experimental wells (with a dedicated tool to make reproducible and small wounds); two wells per case are dedicated to cell counting; rinsing with PBS and an addition of culture medium with or without the peptide according to the invention depending on the case, in the presence or absence of Propionibacterium acnes (100 bacteria for 1 HaCaT cell) are carried out. Photos are taken (to calculate the area of the wound at T0 over a specific area). The cultures are stopped after 24 hours of incubation and photos are taken on the same areas as at T0. A photo processing software (Image J) can calculate for each case the area at T0 and the area recolonized by cells, 24 hours after injury, and establish a ratio.

2—Results

2.1—Action at the Level of the Epidermis

2.1.1—Improvement of the Epidermal Barrier and Harmonization of the Maturation of the Epidermis

The horny layer (also called cornified layer or envelop, or stratum corneum) is an assembly of great complexity associating, on the one hand, cells without nucleus, flat and strongly linked to each other and, on the other hand, lipids and proteins whose composition and assembly provide the unique properties of this structure very resistant to physical, chemical and biological attacks from the environment.

The keratinocytes gradually mature by acquiring a very resistant outer shell formed of proteins called involucrin, loricrin and periplakin, linked together thanks to the intervention of transglutaminases, enzymes sensitive to calcium. In addition, SPRRs (Small Proline Rich Region Proteins), other proteins involved in the maturation and homeostasis of the stratum corneum, serve to strengthen this protein shell by creating flexible but resistant bridges between proteins, there again thanks to the transglutaminase activity. There are also LCEs (Late Cornified Envelope Proteins) which are among the last components to be bridged during the maturation phase. LCEs form a large family largely linked to the epidermis with a structural but also functional role in the control of microbial attacks. LCE3A is notably capable of acting in the control of populations of different skin germs (detailed in point 2.1.3 below). Their action is particularly interesting as it is coupled with a limitation of the anarchic proliferation of keratinocytes, an increase in their maturation in the formation of the stratum corneum and the moderation of inflammatory phenomena, phenomena observed in lesions such as psoriasis, dermatitis, or eczema (detailed in point 2.1.5 below).

Furthermore, ceramides are of great importance in the formation of the stratum corneum and its proteolipid matrix, hence the importance of cosmetic active agents stimulating their synthesis and/or their deposition at the level of the stratum corneum.

A good barrier function is also very dependent on filaggrin, and filaggrin-2 produced by keratinocytes where they undergo significant metabolism. They are used for a time to stabilize the corneocyte by attaching themselves to the keratins.

TABLE 1
Variation compared to the control in the expression of genes encoding proteins of
epidermal differentiation and formation of the stratum corneum (DNA-Microarray)

| Expressed gene | Variation | Name and function of the protein |
|---|---|---|
| LOR | ×193 | Loricrin: major protein of the stratum corneum which confers |
| | | its cohesion/rigidity; linked to the other proteins of the layer by |
| | | the enzymatic action of TGM3 and TGM1 (see below). |
| IVL | ×8.43 | Involucrin: major protein also of the stratum corneum; confers |
| | | its cohesion/rigidity; linked to other proteins in the stratum |
| | | corneum by the enzymatic action of TGM1 (see below). |
| PPL | ×4.12 | Periplakin: serves as a link between the desmosome and the |
| | | stratum corneum; interacts with the intermediate filaments. |
| TGM1 | ×4.66 | Transglutaminase-1: enzyme that binds envoplakin, periplakie |
| | | and involucrin to form the stratum corneum. These crosslinks |
| | | between different structural proteins of the stratum corneum |
| | | ensure rigidity by relying on desmosomes. This same enzyme |
| | | catalyzes the attachment of these units to those produced by |
| | | TGM3 (see below). In addition, it catalyzes the attachment of w- |
| | | hydroxyceramides to proteins already bound together in the |
| | | stratum corneum, which helps maintain hydration of the stratum |
| | | corneum. |
| TGM3 | ×132 | Transglutaminase-3: enzyme binding loricrin to SPRRs (see |
| | | below). |
| TGM5 | ×27.1 | Transglutaminase-5: enzyme that binds envoplakin, periplakin |
| | | and involucrin together to create a rigid structure of the stratum |
| | | corneum. Allows anchoring of these plaques to the desmosomes. |
| SPRR1A | ×2.00 | Small Proline Rich Region Proteins: the proteins of the |
| SPRR2A | ×24.09 | maturation and homeostasis of the stratum corneum, serve to |
| SPRR2C | ×15.35 | strengthen the protein shell of the corneocyte by creating |
| SPRR2E | ×12.48 | flexible but resistant bridges between proteins (loricrin) thanks |
| SPRR2F | ×15.29 | to the activity of transglutaminases. |
| SPRR2D | ×23.97 | |
| SPRR2B | ×451.61 | |
| SPRR2G | ×687.57 | |
| SPRR3 | ×48.62 | |
| SPRR4 | ×23.16 | |
| LCE3A | ×306.86 | Late Cornified Envelop Proteins: they are the last components |
| LCE3B | ×324.01 | to be bridged during the maturation phase in the stratum |
| LCE3D | ×1454.67 | corneum. They are also believed to have a role in antimicrobial |
| LCE3E | ×612.90 | defense. |
| LCE2C | ×37.7 | |
| CRNN | ×258.60 | Cornulin: marker of the terminal epidermal differentiation. |
| | | Strongly reduced in case of eczema. |
| TCHH | ×2.39 | Trichohyaline: structural protein that helps strengthen the |
| | | barrier by cross-bridging with other proteins in the stratum |
| | | corneum using the enzyme TGM1; is used in particular to |
| | | connect the keratins of the intermediate filaments. |
| CALML5 | ×10.73 | Calmodulin-like protein-5: controls the differentiation of the |
| | | epidermis, its inhibition leads to barrier defects and abolishes |
| | | the production of keratohyalin granules. Forms a complex with |
| | | calcium; calmodulins provide control over a large number of |
| | | enzymes, ion channels, aquaporins and other proteins. |
| FLG | ×25.40 | Filaggrin: see above. |
| FLG2 | ×197 | Filaggrine-2: another member of the FLG-Like protein family- |
| | | present on the human epidermal differentiation complex. |
| PRSS8 | ×2.74 | Serine protease-8: essential for the formation of epithelial |
| | | barriers, a co-factor of matriptase; cleaves profillagrin into |
| | | filaggrin and helps hydrate the stratum corneum. |
| ASPRV1 | ×430 | Skin aspartic protease: cleaves profillagrin into filaggrin and |
| | | helps hydrate the stratum corneum; its deficiency also leads to |
| | | the formation of fine wrinkles. |
| PADI-1 | ×185.61 | Peptidyl Arginin Deaminase: serves in the process of |
| | | formation of NMF by deaminating filaggrin. |
| ALOX12B | ×7.92 | Arachidonate-12-lipoxygenase: acts upstream of ALOXE3 on |
| | | the lineolate moiety of linoleyl acyl ceramides (omega- |
| | | hydroxyacyl-sphingosine) to produce an epoxy-ketone |
| | | derivative, a crucial step for omega-hydroxyceramides to bind to |
| | | envelope proteins cornified by transglutaminases; therefore |
| | | plays a crucial role in the synthesis of the lipid envelope of |
| | | corneocytes and in the establishment of the skin barrier. |
| ALOXE3 | ×5.17 | Hydroperoxide isomerase: acts downstream of ALOX12B; |
| | | therefore plays a crucial role in the synthesis of the lipid |
| | | envelope of corneocytes and in the establishment of the skin |
| | | barrier. |
| CERS3 | ×2.38 | Ceramide synthase 3: ceramide synthase which catalyzes the |
| | | formation of ceramides in C24 and those with long acyl chains; |
| | | these ceramides help to maintain the permeability of the skin |
| | | and its water balance. CERS3 is known to increase during |
| | | keratinocyte differentiation. |
| ELOVL4 | ×5.06 | 3-keto acyl-CoA synthase: catalyzes the limiting step of the 4 |
| | | reactions of the long chain fatty acid elongation cycle; formation |
| | | of the corneocyte lipid envelope. |
| ABHD5 | ×2.14 | 1-acylglycerol-3-phosphate O-acyltransferase: this enzyme |
| | | plays a key role in lipid metabolism within the lamellar bodies, |
| | | contributing to the formation of the skin lipid barrier. |
| CDSN | ×29.60 | Corneodesmosin: presence essential for the integrity of the |
| | | epidermal barrier; protein which binds the corneocytes to each |
| | | other while maintaining the flexibility of the stratum corneum |
| | | allowing it not to break under shocks and extensions. |
| OCLN | ×8.65 | Occludin: plays an important role in the formation and |
| | | regulation of the tight junction; capable of inducing adhesion |
| | | when expressed in cells lacking tight junctions. |
| TJP1 | ×2.02 | Tight junction protein ZO-1: structural protein connecting |
| | | tight junction transmembrane proteins such as claudins and |
| | | occludin to the actin cytoskeleton. |
| CGNL1 | ×26.40 | Cingulin-like protein-1: involved in anchoring tight junctions |
| | | to the actin cytoskeleton. |
| CLDN4 | ×10.15 | Claudins: proteins present at the tight junctions that ensure |
| CLDN7 | ×7.24 | cohesion between the corneocytes, via the actin network in the |
| CLDN23 | ×2.69 | upper part of the epidermis; they therefore have a role of |
| | | guardian of water homeostasis, preventing the evaporation of |
| | | water. |
| DEFB103B | ×337.87 | Human beta defensin-3: improves the barrier function via the |
| | | tight junctions, by the CCR6 receptor, but also by a strong |
| | | induction of claudins (see in this table), aPKC kinases, Rac1 |
| | | essential in the regulation of tight junctions, and GSK3 involved |
| | | in the expression of tight junction proteins. |
| DMKN | ×3.58 | Dermokin: specific glycoprotein of the skin located in the upper |
| | | part of the epidermis. Its experimental deficiency induces a |
| | | scaly skin and fine wrinkles, as well as a disturbance of |
| | | insensible water loss; the stratum corneum is weakened with in |
| | | particular less ceramides present; it has been described that this |
| | | protein would play a regulatory role in inflammatory |
| | | dyskeratosis (ichthyosis and psoriasis). |

These data show that many genes involved in epidermal differentiation and in the formation of the skin barrier are strongly stimulated by the peptide according to the invention. The peptide acts at all levels: from the metabolism of filaggrin to the production of the stratum corneum elements, the formation of lipidic lamellar bodies, and the formation of tight junctions.

The peptide of the invention thus acts favorably to guarantee the epidermis homeostasis, ensuring a good balance between the renewal by differentiation of the cells and the formation of a skin barrier of effective quality in particular regarding external aggressions. This type of activity profile guarantees good re-epithelialization of the skin tissue in subjects who have experienced an episode of acne.

Equivalent skins were prepared by first manufacturing an equivalent dermis, then once it contracted, human keratinocytes were seeded on the surface of the gel. After 14 days, equivalent skins were obtained, on which carbopol gels containing the peptide of the invention or the solvent alone were applied to their surface. After 2 days of contact, the skins were frozen to be sectioned into thin 7 μm slides and then marked in order to visualize the macrostructure of the layers. A quantification of the thickness of the stratum corneum was carried out on photos using an image software.

TABLE 2
Thickness of the stratum corneum quantified
on photos of equivalent skins

| | Variation (%) |
|---|---|
| Control | Ref |
| 8 ppm of peptide | +150%; p < 0.01 |
| 10 ppm of peptide | +143%; p < 0.05 |

These results show the positive effect of the peptide according to the invention on epidermal differentiation. The peptide significantly increases the thickness of the stratum corneum.

2.1.2—Epidermis Hydration and Smoothing

In addition to proteins allowing the constitution of an effective skin barrier, other proteins of the epidermis help maintain good hydration and smoothing of the skin.

Filaggrin and filaggrin-2, whose importance in the formation of the barrier has been seen, end up being modified and degraded into amino acids by the action of dedicated enzymes encoded by PRSS8, ASPRV1 and PADI-1 giving essential components of the natural moisturizing factor (NMF) found in the stratum corneum.

Hyaluronic acid, synthesized by keratinocytes, provides the epidermis with resistance to skin pressure, so that the skin protects the underlying structures. These properties are due to the unusual three-dimensional structure of hyaluronic acid, which occupies a very large volume relative to its molecular weight. By capturing water, the hyaluronic acid molecule expands and becomes perfectly resistant to compression, thus giving the epidermis elastic properties, ensuring good hydration, and contributing to a smoother skin.

Kallikreins are proteases involved in the renewal of the stratum corneum since they allow natural desquamation, thus ensuring a gentle “natural” smoothing effect. The increased expression and synthesis of these enzymes may enhance the natural process of corneocyte desquamation and be similar to a natural peel. Nevertheless, the desquamation process is managed by a balance between these proteases and their inhibitors. Therefore, it is also interesting to stimulate the expression and synthesis of natural inhibitors of these proteases, such as ELAFIN, for example, which will regulate the desquamation process.

TABLE 3
Variation compared to the control in the expression of genes encoding proteins
involved in hydration and smoothing of the epidermis (DNA-Microarray)

| Expressed gene | Variation | Name and function of the protein |
|---|---|---|
| FLG | ×25.40 | Filaggrin: serves as a substrate for the manufacture of NMF |
| | | (Natural moisturizing factor) |
| PADI1 | ×185.61 | Peptidyl arginin deaminase: serves in the process of |
| | | formation of the NMF by deaminating filaggrin. |
| ASPRV1 | ×430.83 | Skin aspartic protease cleaves profillagrin into filaggrin |
| | | and helps hydrating the stratum corneum; its deficiency |
| | | during aging leads to the formation of fine wrinkles. |
| PRSS8 | ×2.74 | Serine protease 8: cleaves profillagrin into filaggrin and |
| | | helps hydrating the stratum corneum. |
| AQP5 | ×3.11 | Aquaporin 5: a small protein that forms water channels |
| | | across cell membranes and provides selective water |
| | | transport. |
| KLK5 | ×2.2 | Kallikrein 5: serine protease involved in desquamation. |
| PI3 | ×7.73 | Elafin: desquamation modulator peptide. |
| CLDN4 | ×5.48 | Claudins: proteins present at the tight junctions that ensure |
| CLDN7 | ×7.24 | cohesion between the corneocytes, via the actin network in |
| CLDN23 | ×2.69 | the epidermis upper part; they therefore have a role of |
| | | guardian of water homeostasis, preventing the evaporation |
| | | of water. |

These data show that in the presence of the peptide according to the invention, a large number of proteins involved in hydrating and smoothing the skin are strongly expressed.

TABLE 4
Variation compared to the control of the secretion of hyaluronic
acid by cultured keratinocytes/effect of different concentrations
of the peptide according to the invention at 72 hours of contact

| Pal-KTSKS concentration | Variation %/non treated cells |
|---|---|
| 6 ppm | +33%; p < 0.01 |
| 8 ppm | +42%; p < 0.01 |
| 10 ppm | +62%; p < 0.01 |
| 15 ppm | +107%; p < 0.01 |

These data show a dose-dependent increase in the synthesis of hyaluronic acid, in the presence of the peptide according to the invention.

2.1.3—Antimicrobial Action and Skin Immunity

To protect against pathogenic bacteria or bacteria which may lead to undesirable effects, for example Propionibacterium acnes (the acne bacteria) or against yeasts of the Malassezia genus (which causes dandruff), keratinocytes synthesize antimicrobial peptides (AMPs); defensins, cathelicidins, S100 proteins and PI3 are examples of these. By their targeted action against microorganisms harmful to the skin, antimicrobial peptides will protect and strengthen the skin microbiota.

These peptides are important effectors of the “chemical barrier” put in place to protect the skin from infection and the resulting inflammation.

In addition, these peptides act by modulating innate immune responses, to obtain protection against infection, control of inflammation and scarring, and initiate adaptive immune responses. The human beta-defensin-3 (HBD3) for example is an antimicrobial peptide which intervenes at several levels. It is a key molecule in the skin's immune system, a marker of immune barrier function in the skin. In particular, it has a broad spectrum of destruction against gram+ and gram− bacteria and yeasts. Stimulating the expression of this peptide is of great interest in cosmetics, on the one hand in the prevention and treatment of acne (against the Propionibacterium acnes bacterium) and, on the other hand, in the treatment of a dandruff condition. (against yeasts of the genus Malassezia).

TABLE 5
Variation compared to the control in the expression of genes encoding
proteins involved in antimicrobial defense (DNA-Microarray)

| Expressed gene | Variation | Name and function of the protein |
|---|---|---|
| DEFB103B | ×337.87 | Human beta defensin-3: broad spectrum antimicrobial peptide |
| RNASE7 | ×40.85 | RNase 7: exhibits abroad spectrum of antimicrobial activity; one |
| | | of the most powerful and effective human antimicrobial proteins |
| | | known. |
| PI3 | ×7.73 | Elafin: antimicrobial peptide. |
| S100A7 | ×15.73 | Psoriasin: participates in innate immune defense against |
| | | pathogens on the surface of the skin. |
| DEFB1 | ×4.48 | Beta-1-defensin: antimicrobial peptide. |
| LCE3A | ×306.86 | Late Cornified Envelop Proteins: these are the last components |
| LCE3B | ×324.01 | to be bridged during the maturation phase in the stratum corneum. |
| LCE3D | ×1454.67 | They are also reported to have a role in antimicrobial defense. |
| LCE3E | ×612.9 | |
| LCE2C | ×37.7 | |
| HMOX1 | ×29.27 | Heme Oxygenase-1: inducible antioxidant enzyme, cytoprotective |
| | | essential to minimize the effects of oxidative stress in connection |
| | | with NRF2 and Keap1 and against bacteria and other |
| | | microorganisms. |

These data show that in the presence of the peptide according to the invention, a large number of proteins involved in antimicrobial defense are strongly expressed.

TABLE 6
Growth of the P. acnes bacterium: time required to
reach an OD of 1 in the control case and in the presence
of the peptide according to the invention (N =
3 independent experiments; n = 2 cultures per case)

| | | 6 ppm of | 9 ppm of | 12 ppm of |
|---|---|---|---|---|
| Case | Control | peptide | peptide | peptide |
| Time in hours | 26 | 54 | 103 | 135 |

These data show that the peptide according to the invention strongly and dose-dependently inhibits the growth of P. acnes.

This study was also carried out to follow the persistent effect of the peptide according to the invention on P. acnes, and to see if the inhibition can be quickly lifted in the absence of the peptide. The peptide was brought into contact with the cells from the start, as before. After 48 hours of contact and evaluation of the quantity of cells, the cells of all the cases were centrifuged and returned to a fresh medium without solvent or peptide, all the cases having the same quantity of cells. The growth was then assessed again.

As a result, it is observed that the peptide does exert a net effect on the slowing down of growth when it is in the presence of P. acnes and once removed, the cells regain a growth potential identical to what is observed for the control cases. The peptide therefore exhibits an inhibitory effect when in contact with P. acnes and has no persistent effect.

The effect of the peptide on the inhibition of adhesion and biofilm formation of P. acnes was also studied.

When the bacteria growth, they accumulate, and when a bacteria aggregate reaches a sufficient quorum, namely a sufficient cell quantity, a cell behavior change is observed. More intercellular communications between the cells is possible, and the cells modify their physiology, leading to harmful effects for the host, in particular via the production of a protective biofilm. This phenomenon is called “quorum sensing”.

Avoiding the formation of these biofilms by reducing the growth of the germ, or dissociating these biofilms already formed has the advantage of limiting the quorum effect and reducing the pre-inflammatory consequences on the cells of the follicle.

The adhesion of 106 CFU/mL of P. acnes (strain ATCC6919) to a surface for 72 hours in an appropriate medium was evaluated. At the end of the contact plus more or less the peptide, the quantity of isolated cells, not attached, was estimated by an OD at 600 nm.

The biofilm and the bacteria still attached were labeled with crystal violet solution for 20 min. After rinsing, the quantity of crystal violet remaining on the biofilms was assayed, after extraction, also with a measurement of OD at 600 nm.

The results of this study show that in the control cases the bacteria adhere well to the support as a function of time. The OD600 nm goes from 0.0102 to 0.1515, or 14.8 times more than at TO. The peptide can greatly reduce the adhesion of the bacteria at 48 hours and 72 hours, a drop in adhesion of more than 83% is achieved in two concentrations tested. By discouraging the adhesion of P. acnes, the peptide reduces its possibilities of reaching its quorum, this completes the spectrum of action of the peptide according to the invention on P. acnes which has also shown its ability to inhibit its multiplication.

Regarding the formation of the biofilm, it is observed that, in the control case, the biofilm develops from 0.02 units of OD600 nm (24 hours) to 2.7 units, ie 135 times more in 3 days. Contact with the peptide almost completely prevents the formation of the biofilm by P. acnes ((respectively −98% for 6 ppm and −99% for 12 ppm, both p<0.01). By preventing the biofilm formation, the peptide reduces the possibilities of P. acnes to reach a quorum.

The effect of the peptide on the biofilm stability was also evaluated, the peptide being only added 24 hours after the start of the P. acnes culture.

The results show that the biofilm struggles to exist under these conditions. At 72 hours, the biofilm formed, in the control case, is at 1,861 OD600 nm units, a value reduced from 89% to 93% (the two p<0.01 vs the control) for 6 ppm and 12 ppm of peptide respectively. By reducing the stability of the biofilm, therefore reduces the possibilities of P. acnes to reach its quorum.

The lipase activity, ie the ability of P. acnes to metabolize certain lipids supplied by sebum, is also one of the criteria for the birth of the virulence of P. acnes. This has also been tested.

A culture of P. acnes was carried out. The cells were then separated from the culture medium containing the lipases excreted by P. acnes. The peptide of the invention was added to samples of this cell-free medium and lipase activity was monitored using a fluorescent 4-methylumbelliferyl oleate (4-MUO) probe which is converted by lipases to fluorescent 4-methylumbelliferone, and whose signal has been recorded.

TABLE 7
Reduction in lipase activity of P. acnes, effect of the invention
peptide, n = 6. The means are in relative fluorescence units

| | | 6 ppm of | 9 ppm of | 12 ppm of |
|---|---|---|---|---|
| | Solvent control | peptide | peptide | peptide |
| Variation (%) | Reference | −22%; p < 0.01 | −64%; p < 0.01 | −88%; p < 0.01 |

The results show that the peptide exhibits a dose-dependent inhibitory effect on the activity of the extracellular lipases of P. acnes.

It is also interesting to mention that the peptide according to the invention is selective towards P. acnes. The following test on S. epidermidis was carried out for this purpose. S. epidermidis is one of the most common commensal germs on skin. It serves as a repellent, in particular for pathogenic S. aureus, and is said to control the excessive expansion of P. acnes. It is therefore interesting to see that the peptide according to the invention depresses the growth of this germ, which would otherwise indicate a lack of selectivity and the risk of dysbiosis.

S. epidermidis was inoculated in an appropriate medium and its growth was followed over time. The peptide according to the invention (6 ppm and 12 ppm) was tested on these cells for 48 hours in comparison with the control case (n=6).

The results showed that the growth of S. epidermidis proceeds as expected in the control case. At the same time, the peptide of the invention only induces a slight, temporary slowdown in growth relative to control.

An in vivo study on 30 volunteers conforted these results by showing that the peptide according to the invention did not vary S. epidermidis neither over time (28 days) with the cream according to the invention nor compared to placebo.

TABLE 8
Effect of Propionibacterium acnes on keratinocyte migration.
Variation compared to the control of keratinocyte migration
in the presence of Propionibacterium acnes (N =
3 independent experiments, n = 2 cultures per case)

| | | 5 ppm of | 7 ppm of |
|---|---|---|---|
| Case | Control | peptide | peptide |
| Without P. acnes | Reference 1 | −3% (nsd) | −0.36% (nsd) |
| With P. acnes | +106.4 | | |
| | Reference 2 | −49.67% | −66.15% |
| | | (p < 0.01) | (p < 0.01) |

These data show that the acne bacteria strongly promote the migration of keratinocytes (+106.4%), as described in the scientific literature. In the absence of Propionibacterium acnes, the peptide according to the invention has no effect on the migration of keratinocytes. In the presence of Propionibacterium acnes, the peptide according to the invention slows down the migration of keratinocytes in a dose-dependent and significant manner. As a result, it will slow down the development of acne and reduce its severity.

2.1.4—Detoxifying Action

TABLE 9
Variation compared to the control of the gene
expression encoding proteins involved in the
detoxification of the epidermis (DNA-Microarray)

| Expressed gene | Variation | Name and function of the protein |
|---|---|---|
| HMOX1 | ×29.27 | Heme Oxygenase 1: antioxidant enzyme, |
| | | cytoprotective against the effects of |
| | | oxidative stress. |

These data show that the peptide according to the invention strongly stimulates the Heme Oxygenase 1 enzyme which acts in the defense against oxidation (protection against reactive oxygen species).

2.1.5—Action Intended to Reduce the Deleterious Effects of Inflammation

Recently, it appeared that antimicrobial defense peptides had a broader action in the skin and that they also had a role in cell proliferation, migration, and differentiation, in the regulation of inflammatory responses by controlling the production of different cytokines, in the development of wound healing in the epidermis and in the improvement of the barrier function. This is particularly the case with human beta defensin-3. It is further described as involved in skin immunity via the production of various cytokines/chemochines. Furthermore, it is also described to counteract the inflammatory effects of bacterial lipopolysaccharide. Finally, it has a feedback control of inflammatory activity by inhibiting the TLRs (Toll Like receptor) pathway.

The skin is subjected to constant stress (exposure to UV rays, smoke, pollutants, etc.) some of which provoke a direct or indirect inflammatory response. The uncontrolled or constant inflammatory response, although of low intensity, results in the production of cytokines such as IL-1α, IL-1β, IL-6, TNF α, and active lipids such as PGE2 intended to attract or stimulate the production of a pro-inflammatory secretome from other cells, causing cascade reactions. The pro-inflammatory microenvironment thus formed leads to modifying the skin homeostasis and gradually leads to the modification or even destruction of the biomolecules of cells and tissues. It also leads to the disruption of the integrity of the skin barrier. Thus, the mediators of inflammation, IL-6 and PGE-2, are known to induce, via micro-inflammations, the phenomena of premature aging. In addition, sensitive and irritated skin is characterized by an abnormally high secretion of cytokines, pro-inflammatory peptides (IL-1, IL-6 for example) and pro-inflammatory lipids (PGE-2 for example).

TABLE 10
Variation compared to the control in the expression of genes encoding proteins involved
in the protection of the skin against inflammatory phenomena (DNA-Micoarray)

| Expressed gene | Variation | Name and function of the protein |
|---|---|---|
| DEFB103B | ×337.87 | Human beta defensin-3: see its action above. |
| IL37 | ×7.82 | Interleukin-37: anti-inflammatory cytokine, suppresses or |
| | | reduces the production of pro-inflammatory cytokines, |
| | | including IL1α and IL6; known to suppress keloid |
| | | fibroblasts; inhibits NF-κB and mitogen activation protein |
| | | kinase; “Therapeutic cytokine” against inflammatory |
| | | disorders including psoriasis. |
| SPINK5 | ×3.77 | Serine protease inhibitor Kazal-type 5: serine protease |
| | | inhibitor, important for anti-inflammatory protection of the |
| | | skin; contributes to the integrity and barrier function of the |
| | | skin by regulating the activity of proteases involved in the |
| | | desquamation and defense of the skin. |
| HMOX1 | ×29.27 | Heme Oxygenase-1: strong anti-inflammatory activity, in |
| | | particular by its action against bacterial lipopolysaccharide. |
| PRSS8 | ×2.74 | Serine protease-8: works by reducing the pro-inflammatory |
| | | phenomena associated with TLR4, the activation of which |
| | | leads to an intracellular NFB signaling pathway and the |
| | | production of inflammatory cytokines. |
| TIMP1 | ×3.68 | Metallopeptidase inhibitor 1: inhibitor of metalloproteases, |
| | | thus limits the degradation of collagen. |
| TIMP2 | ×5.96 | Metallopeptidase inhibitor 2: inhibitor of metalloproteases, |
| | | thus limits the degradation of collagen. |
| ANXA1 | ×3.05 | Annexin A1: has anti-inflammatory activity. It binds to |
| | | membrane phospholipids, contributing to their repair. Inhibits |
| | | the formation of PGE2 and the activation of NFκB. |
| LCE3A | ×306.86 | Late Cornified Envelop 3A: plays a positive role against |
| | | inflammation. |

These data show an activity against inflammation through the overexpression of various genes, including that encoding the cytokine IL-37, that encoding human beta-defensin 3 and that encoding annexin A1. Thus, the peptide according to the invention, incorporated into a cosmetic composition, being capable of strongly stimulating the gene expression of human beta defensin 3, an integral part of the innate immune system, will have a defensive action to prevent the penetration or the proliferation of infectious agents in the skin and the resulting inflammation. This is reinforced by the results of ELISA tests described below showing a decrease in production of two markers of inflammation in the human keratinocyte: basal for PGE2, after UVB exposure for PGE2 and IL6.

TABLE 11
Variation compared to the control of the secretome
of pro-inflammatory mediators by NHKs exposed
to UVB (by immune-enzymatic assay)

| Marker | 3 ppm | 10 ppm | 15 ppm |
|---|---|---|---|
| IL6 | −4% (nsd) | −33% (p < 0.01) | −65% (p < 0.01) |
| PGE2 | +1% (nsd) | −41% (p < 0.01) | −63% (p < 0.01) |

nsd: non significative

These data show the very interesting effect of the peptide according to the invention to moderate the secretion of pro-inflammatory mediators by NHKs which have been exposed to UVB irradiation. This effect is particularly interesting for sensitive skin irritated by exposure to radiation and various pollutants that cause micro-inflammation.

The preferred peptide according to the invention improves the cutaneous defense system against bacteria, oxidants, and radiation by modulating the inflammatory phenomena which result from these toxic agents. All the presented markers act in this direction.

2.2—Action on the Dermal Extracellular Matrix and on the Basement Membrane

In addition, advantageously, the tests given below show that the peptide according to the invention also exhibits a complementary activity on the dermal tissue and its essential matrix components (collagens, elastin, fibronectin).

The dermis of an 80-year-old has four times more fragmented collagen than that of people 20-30 years of age who have longer fibers. This fragmentation leads to a reduction of up to 80% of the interactions that cells have with their matrix. With age, dermal fibroblasts produce less supportive protein, including less collagen I, the most abundant protein in the skin, and less elastin. This explains the structural and functional decline of the skin, which becomes less dense, less organized, and less dynamic. The weakening of the qualities of the support tissue causes a decrease in the visco-elastic characteristics of the skin: firmness, elasticity and tone are thus reduced by about 13% per decade.

Collagen 4 is the most important building block of the basement membrane. This membrane provides the junction and separation between the dermis and the epidermis (it is also called the dermo-epidermal junction or DEJ).

TABLE 12
Variation in the production of the C terminal peptide of pro-
collagen-I, collagen-I, collagen-IV, fibronectin in the presence
of the preferred peptide according to the invention:

| | Terminal C | | | |
|---|---|---|---|---|
| | peptide of pro- | | | |
| | collagen I | Collagen I | Collagen IV | Fibronectin |
| | variation %/ | Variation %/ | Variation %/ | Variation %/ |
| Concentrations | control | control | control | control |
| Control | Reference | Reference | Reference | Reference |
| 8 ppm | +61%; p < 0.01 | +75%; p < 0.01 | +18%; p < 0.01 | +40%; p < 0.01 |
| 10 ppm | +94%; p < 0.01 | +184%; p < 0.01 | +98%; p < 0.01 | +67%; p < 0.01 |

The MMPs (dermal matrix proteases) are naturally controlled in tissues by their inhibitors TIMPs (tissue inhibitors of dermal matrix proteases) which prevents blind enzymatic degradation activity. It is the balance of TIMP versus MMP that determines the level of activity of the latter. TIMPs are small glycoproteins whose production is associated with the reduction of chronic pathologies linked to MMPs and a reduction in photo-damage linked to UV rays. By combining with MMPs, TIMPs neutralize them and therefore limit the fragmentation of the dermal matrix, which thus preserves its elasticity and firmness.

TABLE 13
Variation compared to the control in the expression
of genes encoding TIMPs involved in the protection
of the dermis (DNA-Microarray)

| Expressed gene | Variation | Name and function of the protein |
|---|---|---|
| TIMP1 | ×3.68 | Metallopeptidase inhibitor 1: metalloprotease |
| | | inhibitor, thus limiting collagen degradation. |
| TIMP2 | ×5.96 | Metallopeptidase inhibitor 2: metalloprotease |
| | | inhibitor, thus limiting collagen degradation. |

These data show that the peptide according to the invention can, in addition to its activity on the epidermis, strengthen the dermis and the basement membrane, for more skin firmness and elasticity.

2.3—Beneficial Action on Nails and Hair

2.3.1—on Nails

Filaggrin and trichohyalin coexist with keratins 6 and 16 in the nail bed. These are constituent proteins. Filaggrin and trichohyalin may act to stabilize the network of intermediate filaments of K6 and K16.

TABLE 14
Variation compared to the control in the expression of genes encoding constituent
proteins of the nail or involved in its formation (DNA-Microarray)

| Expressed gene | Variation | Name and function of the protein |
|---|---|---|
| TCHH | x 2.39 | Trichohyalin: present in the nail bed |
| FLG | x 25.40 | Filaggrin: also present in the nail bed |
| PPL | x 4.12 | Periplakin: serves as a link between the desmosome and the |
| | | horny envelope; interacts with the intermediate filaments; |
| | | confers mechanical resistance to nail-like hard epithelia. |

These data show that two constituent compounds of the nail and a compound involved in its formation or retention have their gene expression increased following contact with the peptide according to the invention.

The compound according to the invention thus appears to be perfectly indicated for the maintenance of healthy nails or the treatment of damaged nails.

2.3.2—on Hair (Comprising Eyelashes and Eyebrows)

Trichohyalin is expressed in specific epithelia, exceptionally strong mechanically, such as cells in the inner sheath of the hair follicle. It is subjected to modifications by enzymes, in particular transglutaminases, which introduce intra- and inter-proteic crosslinks. It is a reticulated multifunctional protein that functions in the hair's inner root sheath, imparting and coordinating mechanical strength between the peripheral cell envelope structures and the cytoplasmic keratin filament network (intermediate filaments).

TABLE 15
Variation compared to the control in the expression of genes coding for constitutive
proteins of the hair or involved in its formation (DNA-Microarray)

| Expressed gene | Variation | Name and function of the protein |
|---|---|---|
| TCHH | x 2.39 | Trichohyalin: present in the inner sheath of the hair follicle. |
| CDSN | x 29.60 | Corneodesmosin: essential presence for the epidermal barrier |
| | | integrity; protein which binds firmly the corneocytes to each |
| | | other while maintaining the flexibility of the stratum corneum |
| | | which prevents it from breaking under shocks and extensions. |
| | | The absence of corneodesmosin leads to thinning of the hair |
| | | and their loss. |
| ASPRV1 | x 430 | Skin aspartic protease: present in the inner sheath of the hair |
| | | follicle; cleaves profillagrin into filaggrin. |
| ALOX12B | x 7.92 | Arachidonate 12-lipoxygenase: acts upstream of ALOXE3 |
| | | on the lineolate moiety of linoleyl acyl ceramides (omega- |
| | | hydroxyacyl-sphingosine) to produce an epoxy-ketone |
| | | derivative, a crucial step for omega-hydroxyceramides to bind |
| | | to keratin-associated proteins (KAPs) thanks to |
| | | transglutaminases; therefore, plays a crucial role in the |
| | | synthesis of the cell lipid envelope of the root sheath of the |
| | | follicle. |
| ALOXE3 | x 5.17 | Hydroperoxide isomerase: acts downstream of ALOX12B; |
| | | therefore plays a crucial role in the synthesis of the cell lipid |
| | | envelope of the root sheath of the follicle. |
| TGM3 | x 132 | Transglutaminase 3: is expressed in the hair shaft; |
| | | participates in the progressive scaffolding of the hair shaft by |
| | | creating bonds between the intermediate filaments and to the |
| | | proteins associated with keratin (KAPs); thus, contributes to |
| | | the physical resistance of the hair structure. |
| PPL | x 4.12 | Periplakin: serves as a link between the desmosome and the |
| | | hair follicle; interacts with the intermediate filaments; confers |
| | | mechanical resistance to hard epithelia such as the inner |
| | | sheath of the hair follicle. |

These data show that a certain number of compounds of the hair itself and of compounds involved in its formation or maintenance have their gene expression increased following contact with the peptide according to the invention. The compound according to the invention thus appears to be perfectly indicated for action in the capilar field to regulate hair growth, improve quality and appearance, correct defects.

II—on Other Preferred Peptides of the Invention: The Pal-KTSKT (SEQ ID No 7), the Pal-GKTSKS (SEQ ID No 6) and the Pal-KTSKSA (SEQ ID No 8)

1—P. acnes growth curve (same protocol as described above at point 1.4)

P. acnes is thawed and grown over 3 days in M20 media. Then, optical density at 600 nm (OD600) is measured and 2×106 CFU*/mL are plated in well plates. P. acnes is then treated with 6 ppm, 9 ppm and 12 ppm of peptides. To follow bacterial cell multiplication, OD600 is measured regularly for about 1 week. These data are used to generate P. acnes growth curve and to estimate the effect of the peptides of the invention on C. acnes. Each result is a mean obtained from 6 replicates (n=6) and a standard deviation of the mean (sdm) is obtained from these results. Statistical evaluations are performed on results using a Student t test. Two independent experiments are performed (N=2). The time needed for P. acnes to reach 0.5 of OD at 600 nm after treatment with each peptide is shown in the table below.

TABLE 16

| Peptide of the | Time (in hour) to reach OD_{600 nm} = 0.5 +/−sdm | | | |
|---|---|---|---|---|
| invention | Vehicle Control | 6 ppm | 9 ppm | 12 ppm |
| Pal-KTSKS-OH | 43.9 h | 48.8 h** | 55.0 h** | 87.3 h** |
| | +/−0.73 | +/−1.07 | +/−0.31 | +/−1.69 |
| Pal-GKTSKS-OH | 43.9 h | 45.6 h* | 56.2 h** | 70.2 h** |
| | +/−0.73 | +/−1.27 | +/−0.54 | +/−1.70 |
| Pal-KTSKSA-OH | 42.4 h | 47.3 h** | 57.9 h** | 60.5 h** |
| | +/−0.46 | +/−0.68 | +/−0.55 | +/−0.59 |
| Pal-KTSKT-OH | 42.4 h | 47.7 h** | 55.2 h** | 85.1 h** |
| | +/−0.46 | +/−0.88 | +/−0.6 | +/−2.13 |

*p < 0.05 or
**p < 0.01 vs vehicle

The results show that vehicle required 42-44 h for reaching an OD₆₀₀ₙₘ of 0.5.

These results confirm the results given before on the Pal-KTSKS: the peptide triggers a delay in P. acnes growth versus vehicle whatever the concentration used; this effect is dose-dependent of the concentrations (for 6 ppm, 9 ppm and 12 ppm: +5 h, +11 h and +43 h respectively).

The Pal-GKTSK triggers a delay in P. acnes growth versus vehicle; this effect is dose-dependent of the concentration (for 6 ppm, 9 ppm and 12 ppm: +2 h, +12 h and +26 h respectively).

The Pal-KTSKSA triggers a delay in P. acnes growth versus vehicle; this effect is dose-dependent of the concentration (for 6 ppm, 9 ppm and 12 ppm: +5 h, +15 h and +18 h respectively).

The Pal-KTSKT triggersa delay in P. acnes growth versus vehicle; this effect is dose-dependent of the concentration (for 6 ppm, 9 ppm and 12 ppm: +5 h, +13 h and +43 h respectively).

2—Keratinocyte Differentiation

Normal human keratinocytes (NHK) are grown on wells plate. When subconfluence was reached, cells received various concentrations of the peptides. After few days, evaluation of differentiation is compared to solvent control under microscope and cells are fixed to allow the study of involucrin by immunohistochemistry.

The below table summarizes a visual evaluation of keratinocytes differentiation.

TABLE 17

Visual evaluation of NHK differentiation

| | Day 2 | Day 4 |
|---|---|---|
| Pal-KTSKS | + | ++ |
| Pal-KTSKSA | + | ++ |
| Pal-KTSKT | + | ++ |

Results on the Pal-KTSKS are confirmed: the peptide strongly increases keratinocytes differentiation both at day 2 and day 4 after contact.

The results show that the Pal-KTSKSA induces keratinocytes differentiation after 2 and 4 days in a similar way than the Pal-KTSKS.

The results show a similar induction of keratinocytes differentiation of the Pal-KTSKT compared to Pal-KTSKS.

3—Involucrin Quantification

Involucrin is an early marker of keratinocytes differentiation. The below table show the percentage of variation of involucrin vs. Control.

TABLE 18

| | | Involucrin (% var vs. Control); t.test |
|---|---|---|
| Control | — | Ref |
| Pal-KTSKS | 5 ppm | +177%; p < 0.01 |
| Pal-KTSKSA | 5 ppm | +152%; p < 0.01 |
| Pal-KTSKT | 5 ppm | +125%; p < 0.01 |

These results confirm the induction of keratinocytes differentiation for the three peptides according to the invention: the Pal-KTSKS, the Pal-KTSKSA, and the Pal-KTSKT.

D—Galenic/Preparation of a Composition According to the Invention for an End User

The peptide(s) according to the invention can be formulated with additional active cosmetic ingredients, coming if required in support and/or in complement of activity, either in the ingredient form, or at the time of the realization of the final cosmetic composition for the end consumer. This composition can be applied to the face, body, neckline, scalp, hair (from scalp and body, comprising eyelashes and eyebrows), in any form or vehicles known to those skilled in the art, in particular in the form of solution, dispersion, emulsion, paste or powder, individually or as a premix or be vehiculed individually or as a premix.

In cosmetics, applications can be offered in particular in the skin care ranges for the face, body, hair (from scalp and body comprising eyelasches and eyebrows) and in make-up-care ranges. These ingredients can be of any category according to their role(s), the place of application (body, face, neck, bust, hands, hair, etc.), the desired final effect and the targeted consumer, for example antioxidant, tensor, moisturizer, nourishing, protective, smoothing, remodeling, volumizing (lipofiling), acting on the radiance of the complexion, acting on spots, under eye dark circles and bags, anti-glycation, anti-aging, anti-wrinkle, slimming, soothing, myo-relaxing, anti-redness, anti-stretch marks, sunscreen, etc.

The Personal Care Products Council (International cosmetic ingredient dictionary & handbook (20th Ed. 2020) published by “the Cosmetic, Toiletry, and Fragrance Association, Inc.”, Washington, D.C.) describes a wide variety, without limitation, of cosmetic ingredients usually used in the skincare and scalp care industry, which are suitable for use as additional ingredients in the compositions of the present invention.

In particular, mention may be made of at least one of the compounds chosen from compounds of vitamin B3, compounds such as niacinamide or tocopherol, retinoid compounds such as retinol, hexamidine, α-lipoic acid, resveratrol or DHEA, hyaluronic acid, peptides, in particular N-acetyl-Tyr-Arg-O-hexadecyl ester, Pal-KTTKS (SEQ ID No 1), Pal-VGVAPG (SEQ ID No 3), Pal-KTFK (SEQ ID No 9), Pal-GHK, Pal-KMO₂K (MO₂ corresponding to a methionine sulfone), Pal-GQPR (SEQ ID No 2) and Pal-K(P)HG (K(P) corresponding to a proline grafted on the lysine), which are classic active ingredients used in topical compositions cosmetics or dermo-pharmaceuticals. Other additional skin care actives that are particularly useful can be found in Sederma's and Crodarom's commercial literature and at www.croda.com.

In activity reinforcing on the properties of epidermis and/or stratum corneum, the additional active agent can be chosen from the group comprising: phospholipids, various ceramides, sphingosine, phytosphingosine, glycosphingolipids, cholesterol and its derivatives, sterols (in particular those of canola and soybean), fatty acids (in particular linoleic acid, palmitic acid, lipoic acid and thioctic acid), squalane (in particular from olives), triglycerides (in particular coconut oil), lanolin, lanolin alcohols, lanosterol, vitamin D3, tocopheryl nicotinate, various oils (in particular, argan, rose, and baobab), ascorbic acid, N-acetyl cysteine and N-acetyl-L-serine, vitamin B3 compounds (such as niacinamide and nicotinic acid), panthenol, pseudofilaggrine, arginine, serine, salts of PCA (pyrrolidone carboxylic acid), an extract of Centella asiatica leaf (titrated in madecassoside and asiaticoside), certain extracts of plants (wild yam roots, chestnut, cedar bud and solanaceae), plankton and yeast.

Mention may also be made of the actives marketed by Sederma: Venuceane™ (extract of Thermus thermophilus fermentation medium), Moist 24™ (hydroglycolic extract of Imperata cylindrica root), Dermaxyl™ (combination of ceramide 2 and the Pal-VGVAPG peptide), Senestem™ (a Plantago lanceolata cell culture extract), Ceramide 2™ (ceramide), Ceramide HO3™ (hydroxyceramide), Optim Hyal™ (acetylated glucuronic acid oligosaccharides), Meiritage™ (combination of root extracts of Bupleurum falcatum, Astragalus membranaceus and Atractylodes macrocephala) Revidrat™ (myristyl phosphomalate), Pacifeel™ (a Mirabilis jalapa plant extract), Hydronesis™ (fermentation of Salinococcus hispanicus), Shea unsaponifiable NG™, Citystem™ (a cell culture extract ofMarrubium vulgare) and the Pal-KTFK peptide, in particular in its vehiculed form in the commercial product Crystalide™.

In general, the following commercial actives may also be mentioned by way of example: betain, glycerol, Actimoist Bio 2™ (Active organics), AquaCacteen™ (Mibelle AG Cosmetics), Aquaphyline™ (Silab), AquaregulK™ (Solabia), Carciline™ (Greentech), Codiavelane™ (Biotech Marine), Dermaflux™ (Arch Chemicals, Inc), Hydra′Flow™ (Sochibo), Hydromoist L™ (Symrise), RenovHyal™ (Soliance), Seamoss™ (Biotech Marine), Argireline™ (trade name for acetyl hexapeptide-3 of Lipotec), le spilanthol or an extract of Acmella oleracea known under the trade name Gatuline Expression™, an extract of Boswellia serrata under the trade name Boswellin™, Deepaline PVB™ (Seppic), Syn-AKE™ (Pentapharm), Ameliox™, Bioxilift™ (Silab), PhytoCellTec™Argan (Mibelle), Papilactyl D™ (Silab), Preventhelia™ (Lipotec), or one or more active ingredient following sold by Sederma: Subliskin™, Venuceane™, Moist 24™, Vegesome Moist 24™, Essenskin™ Juvinity™, Revidrat™, Resistem™, Chronodyn™, Kombuchka™, Chromocare™, Calmosensine™ Glycokin factor S™, Biobustyl™, Idealift™, Ceramide 2™, Ceramide A2™, Ceramide HO3™ Legance™, Intenslim™, Prodizia™, Beautifeye™, Pacifeel™, Zingerslim™, Meiritage™, Sebuless™, Apiscalp™, Rubistem™, Citystem™, Neonyca™, NG Shea Unsaponifiable™ Majestem™, Hydronesis™, Poretect™, Amberstem™, Synchrolife™, Feminage™, or mixture thereof. Among plant extracts (in the form of classical plant extracts or prepared by an in vitro process) can be used as additional actives, there may more particularly be mentioned extracts of ivy, for example English Ivy (Hedera helix), of Bupleurum chinensis, of Bupleurum falcatum, of arnica (Arnica montana L), of rosemary (Rosmarinus officinalis N), of marigold (Calendula officinalis), of sage (Salvia officinalis L), of ginseng (Panax ginseng), of gingko biloba, of St.-John's-Wort (Hyperycum perforatum), of butcher's-broom (Ruscus aculeatus L), of European meadowsweet (Filipendula ulmaria L), of big-flowered Jarva tea (Orthosiphon stamincus benth), of artichoke (Cynara scolymus), of algae (Fucus vesiculosus), of birch (Betula alba), of green tea, of cola nuts (Cola nipida), of horse-chestnut, of bamboo, of Centella asiatica, of heather, of fucus, of willow, of mouse-ear, of escine, of cangzhu, of Chrysanthellum indicum, of the plants of the Armeniacea genus, Atractylodis platicodon, Sinnomenum, pharbitidis, Flemingia, Coleus comme C. forskohlii, C. blumei, C. esquirolii, C. scutellaroides, C. xanthantus and C. barbatus, such as the extract of root of Coleus barbatus, extracts of Ballote, of Guioa, of Davallia, of Terminalia, of Barringtonia, of Trema, of Antirobia, Cecropia, Argania, Dioscoreae such as Dioscorea opposita or Mexican, extracts of Ammi visnaga, of Siegesbeckia, in particular Siegesbeckia orientalis, vegetable extracts of the family of Ericaceae, in particular bilberry extracts (Vaccinium angustifollium) or Arctostaphylos uva ursi, aloe vera, plant containing sterols (e.g., phytosterol), Manjistha (extracted from plants of the genus Rubia, particularly Rubia cordifolia), and Guggal (extracted from plants of the genus Commiphora, particularly Commiphora mukul), kola extract, chamomile, red clover extract, Piper methysticum extract (Kava Kava™ from Sederma), Bacopa monieri extract (Bacocalmine™ from Sederma) and sea whip extract, extracts of Glycyrrhiza glabra, of mulberry, of melaleuca (tea tree), of Larrea divaricata, of Rabdosia rubescens, of Euglena gracilis, of Fibraurea recisa Hirudinea, of Chaparral Sorghum, of sun flower extract, of Enantia chlorantha, of Mitracarpe of Spermacocea genus, of Buchu barosma, of Law sonia inermis L., of Adiantium capillus-veneris L., of Chelidonium majus, of Luffa cylindrica, of “Japanese Mandan” (Citrus reticulata Blanco var. unshiu), of Camelia sinensis, ofImperata cylindrica, of Glaucium Flavum, of Cupressus sempervirens, of Polygonatum multi/Thrum, of Loveyly hemsleya, of Sambucus nigra, of Phaseolus lunatus, of Centaurium, of Macrocystis pyrifera, of Turnera diffusa, of Anemarrhena asphodeloides, of Portulaca pilosa, of Humulus lupulus, of Coffea arabica, of Rex paraguariensis, or of Globularia cordifolia, of Oxydendron arboretum, ofAlbizzia julibrissin, of Zingimber zerumbet smith, of Astragalus membranaceus, of Atractylodes macrocephalae, of Plantago lanceolata, of lentopodium alpinum (or edelweiss), of Mirabilis jalapa, of Apium graveolens, of Marrubium vulgare, Buddleja davidii Franch, Syringa vulgaris or orchids.

The compositions of the present invention may include other peptides, including, without limitation, di-, tri-, tetra-, penta-and hexapeptides and their derivatives. According to a particular embodiment, the concentration of the additional peptide(s), in the composition, ranges from 1×10⁻⁷% and 20%, preferably from 1×10⁻⁶% and 10%, preferably between 1×10⁻⁵% and 5% by weight. The term “peptide” refers here to peptides containing 10 amino acids or less, their derivatives, isomers and complexes with other species such as a metal ion (e.g. copper, zinc, manganese, magnesium, and others). The term “peptides” refers to both natural peptides and (bio)synthetic peptides. It also refers to compositions that contain peptides and which are found in nature, and/or are commercially available.

Suitable dipeptides for use herein include but are not limited to Carnosine (βAH), YR, VW, NF, DF, KT, KC, CK, KP, KK, TT, PA, PM or PP.

Suitable tripeptides for use herein include, but are not limited to RKR, HGG, GKH, GHK, GGH, GHG, KGH, KHG, KFK, KAvaK, KβAK, KAbuK, KAcaK, KPK, KMOK, KMO₂K (MO₂ being a di-oxygenated sulfoxide methionine), KVK, PPL, PPR, SPR, QPA, LPA, SPA, K(Ac)HG or K(Ac)GH, K(Ac) being a lysine with the amine function of the lateral chain acetylated, as disclosed in WO2017/216177, K(P)HG or K(P)GH, K(P) being a lysine with its lateral chain grafted with a proline, K(Pyr)HG or K(Pyr)GH, K(Pyr) being a lysine with its lateral chain grafted with a pyroglutamic acid, K(Hyp)HG or K(Hyp)GH, K(Hyp) being a lysine with its lateral chain grafted with a hydroxyproline, as disclosed in WO2016/097965.

Suitable tetrapeptides for use as additional peptides herein include but are not limited to GQPR (SEQ ID No 10), RSRK (SEQ ID No 11), KTFK (SEQ ID No 12), KTAK (SEQ ID No 13), KAYK (SEQ ID No 14), KFYK (SEQ ID No 15), or TKPR (SEQ ID No 16).

A suitable non limitative example of pentapeptide is the KTTKS (SEQ ID No 17), and suitable examples of hexapeptides are the GKTTKS (SEQ ID No 18) and VGVAPG (SEQ ID No 19).

Other suitable peptides for use according to the present invention can be selected, this list being not limitative, from: lipophilic derivatives of peptides, preferably palmitoyl (Pal) derivatives or myristoyl (Myr), and metal complexes as aforementioned (e.g. copper complex of the tripeptide HGG or GHK).

Preferred dipeptides include for example N-Palmitoyl-β-Ala-His, N-Acetyl-Tyr-Arg-hexadecylester (Calmosensine™, Idealift™ from Sederma), Pal-RT or Pal-KT (from Sederma). Preferred tripeptide derivatives include for example Pal-GKH and Pal-GHKou N-Biot GHK (from Sederma), the copper derivative of HGG (Lamin™ from Sigma), Lipospondin (N-Elaidoyl-KFK) and its analogs of conservative substitution, N-Acetyl-RKR—NH₂ (Peptide CK+), N-Biot-GHK (from Sederma), Pal-KAvaK, Pal-KβAlaK, Pal-KAbuK, Pal-KAcaK, or Pal-KMO₂K (Matrixyl®synthe′6® from Sederma), Pal-KVK (Syn-Coll™ of DSM), and derivatives thereof. Mention may also be made here of the anti-aging tripeptides of general Formula X-Pro*-Pro*-Xaa-Y described in WO2015181688 application with Xaa selected from Leu, Arg, Lys, Ala, Ser, and Asp, at the N-terminus, X chosen from H, —CO—R¹ and —SO₂-R¹ and at the C-terminal end Y chosen from OH, OR¹, NH₂, NHR¹ or NR¹R², R¹ and R² being, independently of one another, chosen from a alkyl, aryl, aralkyl, alkylaryl, alkoxy and aryloxy group, which may be linear, branched, cyclic, polycyclic, unsaturated, hydroxylated, carbonylated, phosphorylated and/or sulfurized, said group possibly possessing in its backbone a heteroatom particularly O, S and/or or N, and Pro* corresponding to Proline, an analogue or derivative thereof; comprising, for example, Myr-PPL-OH and Myr-PPR—OH.

Here can further be cited also the propigmenting and/or pro-mec dipeptides and tripeptides of general Formula X—(Xaa1)n—Pro*—Xaa2—Y disclosed in WO2014/080376, with n=0, 1 or 2, Xaa1 an hydrophobic aminoacid selected from Ala, Val, Met, Leu, Iso, Phe, Pro, and analogs and derivatives thereof, or a polar aminoacid selected from Ser, Thr, Tyr, Asp, Glu and analogs and derivatives thereof; and when n=2 the two aminoacids Xaa1 being the same or different; Xaa2 being an hydrophobic aminoacid selected from Ala, Val, Met, Leu, Iso, Phe, and analogs and derivatives thereof, or a basic aminoacid selected from Arg, Lys, His, and analogs and derivatives thereof; at the N terminal end X being selected from H, —CO—R¹ and —SO₂-R¹; at the C terminal end Y being selected from OH, OR¹, NH₂, NHR¹ or NR¹R²; R¹ and R² being, independently from each other, selected from an alkyl, aryl, aralkyl, alkylaryl, alkoxy et aryloxy group, that can be linear, branched, cyclic polycyclic, saturated, unsaturated, hydroxylated, carbonylated, phosphorylated and/or sulfured, said group having or not an O, S and/or N heteroatom in its skeleton and Pro* corresponding to a Proline, analog or derivative thereof; comprising for example the following peptides Pal-SPR—OH, Pal-PPR—OH, Pal-QPA-OH, Pal-LPA-OH, Myr-SPA-OH, Pal-PM-OH, Pal-PA-OH and Pal-PP—OH.

Suitable tetrapeptide derivatives for use as additional peptides according to the present invention include, but are not limited to, Pal-KTFK (SEQ ID No 8) or Ela-KTFK (SEQ ID No 20), Ela-KTAK (SEQ ID No 21), Ela-KAYK (SEQ ID No 22) or Ela-KFYK (SEQ ID No 23).

Suitable pentapeptide derivatives for use as additional peptides herein include, but are not limited to, Pal-KTTKS (SEQ ID No 1) (available as Matrixyl® from Sederma), Pal-YGGFXaa (SEQ ID No 24) with Xaa being Leu or Pro, or mixtures thereof.

Suitable hexapeptide derivatives for use herein include, but are not limited to, Pal-VGVAPG (SEQ ID No 3), Pal-GKTTKS (SEQ ID No 6), Pal-HLDIIXaa with Xaa being Trp, Phe, Tyr, Tic, 7-hydroxy-Tic ou Tpi (SEQ ID No 25) and derivatives thereof. The mixture of Pal-GHK and Pal-GQPR (SEQ ID No 2) (Matrixyl® 3000, Sederma) can also be mentioned.

The following marketed peptides can be mentioned as well as additional active ingredients: Vialox™ (INCI name=Pentapeptide-3 (synthetic peptide comprising alanine, arginine, isoleucine, glycine and proline)), Syn-ake™ (β-Ala-Pro-Dab-NH-Bzl) or Syn-Coll™ (Pal-Lys-Val-Lys-OH) marketed by Pentapharm;

Argireline™ (Ac-Glu-Glu-Met-Gln-Arg-Arg-NH₂ (INCI name=Acetyl hexapeptide-3) (SEQ ID No 26), Leuphasyl™ (Tyr-D-Ala-Gly-Phe-Leu) (SEQ ID No 27), Aldenine™ (Gly-His-Lys), Trylagen™ (INCI name=Pseudoalteromonas Ferment Extract, Hydro lyzed Wheat Protein, Hydro lyzed Soy Protein, Tripeptide-10 Citrulline (reaction product of Citrulline and Tripeptide-10 (synthetic peptide constituted of aspartic acid, isoleucine and lysine)), Tripeptide-1), Eyeseryl™ (Ac-β-Ala-His-Ser-His)(SEQ ID No 28), Serilesine™ (Ser-Ile-Lys-Val-Ala-Val) (SEQ ID No 29) or Decorinyl™ (INCI name: Tripeptide-10 Citrulline=reaction product of Citrulline and Tripeptide-10 (synthetic peptide constituted of aspartic acid, isoleucine and lysine) marketed by Lipotec; Collaxyl™ (Gly-Pro-Gln-Gly-Pro-Gln (SEQ ID No 30)) or Quintescine™ (Cys-Gly) marketed by Vincience;

Cytokinol™LS (casein hydrolysate) marketed by Les Laboratoires Serobiologiques/Cognis; Kollaren™ (Gly-His-Lys), IP2000™ (Pal-Val-Tyr-Val) or Meliprene™ (INCI name=Monofluoroheptapeptide-1: reaction product of acetic acide and a synthetic peptide comprising arginine, glycine, glutamic acid, histidine, norleucine, p-fluorophenylalanine and tryptophan) marketed by l′lnstitut Europeen de Biologie Cellulaire;

Neutrazen™ (Pal-His-D-Phe-Arg-NH₂) marketed by Innovations; or

BONT-L-Peptide™ (INCI name=Palmitoyl Hexapeptide-19: reaction product of palmitic acid and Hexapeptide-19 (synthetic peptide constituted of asparagine, aspartic acid, lysine and methionine), Timp-Peptide™ (INCI name=Acetyl Hexapeptide-20: reaction product obtained by acetylation of Hexapeptide-20 (synthetic peptide constituted of alanine, glycine, lysine, valine and proline) or ECM Moduline™ (INCI name=Palmitoyl Tripeptide-28: reaction product of palmitic acid and Tripeptide-28 (synthetic peptide constituted of arginine, lysine and phenylalanine) marketed by lnfinitec Activos.

It is also possible to combine the invention peptide(s) with one or more cyclic peptides, in particular those extracted from linseed oil described in the Applicant's patent application FR1850845.

Different compositions/formulations according to the invention are described below with examples of additional active ingredients.

The active ingredient according to the invention is as described in point C/comprising 100 ppm of peptide(s) according to the invention.

This ingredient is generally formulated within a range of 1 to 5%, preferably 3%.

1—Cream Form, for Example an Antiageing Day Cream for the Face.

TABLE 19

| Ingredients | INCI name | Role | % |
|---|---|---|---|
| Part A: | | | |
| H₂O | / | / | qsp100 |
| Carbopol ™ Ultrez 10 | Carbomer | Thickener/Gelling agent | 0.30 |
| Part B: | | | |
| Brij S2-SS-(RB) ™ | Steareth-2 | Emulsifier | 0.40 |
| Brij S10-SO-(RB) ™ | Steareth-10 | Emulsifier | 1.20 |
| Crodafos CES-PA-(RB) ™ | Cetearyl Alcohol (and) | Emulsifier/conditionner | 4.00 |
| | Dicetyl Phosphate (and) | | |
| | Ceteth-10 Phosphate | | |
| Crodacol CS90-PA-(RB) ™ | Cetearyl Alcohol | Emollient | 1.50 |
| Laurocapram | Laurocapram | Emollient | 2.50 |
| Crodamol ™ AB-LQ-(RB) | C12-15 Alkyl | Emollient | 1.50 |
| | Benzoate | | |
| Crodamol ™ OSU-LQ-(JP) | Diethylhexyl Succinate | Emollient | 7.00 |
| Part C: | | | |
| Glycerin | Glycerin | Humectant | 2.50 |
| Octanediol | Caprylyl Glycol | Humectant/Emollient | 0.50 |
| Part D: | | | |
| Phenoxyethanol | Phenoxyethanol | Preservative | qs |
| Part E: | | | |
| Potassium sorbate | Potassium Sorbate | Preservative | qs |
| Part F: | | | |
| H₂O | / | / | 4.00 |
| NaOH 30% | Sodium Hydroxide | pH adjuster | 0.40 |
| Part G: | | | |
| Ingredient according to the | / | Active | 3.00 |
| invention | | | |

Example(s) of Additional Active Ingredient(s):
a moisturizing and smoothing ingredient such as: OPTIM HYAL™, marketed Sederma, comprises oligosaccharides of acetylated glucuronic acids having a structure similar to fragments of hyaluronic acid.
a sebum-regulating ingredient such as:
SEBULESS™, marketed by Sederma, comprising a Syringa vulgaris extract obtained by in vitro cell culture, acting as a purifying sebum regulator, mattifying and refreshing complexion, and bluring skin imperfections.
PORETECT™, marketed by Sederma, comprising an association of linseed extract titrated in cylolinopeptides and a celery extract titrated in senkyunolides; this active acts on skin firmness, tone and density, thus strengthening the pore support structures sagging with age.
In reinforcement of the activity: an ingredient acting on the skin elastic properties and the skin barrier such as:
IDEALIFT™, marketed by Sederma, comprising the lipodipeptide N-acetyl-Tyrosyl-Arginyl-O-hexadecyl ester, combating facial flaccidity and improving resistance to gravity, in particular through elastin synthesis stimulation.
DERMAXYL™, marketed by Sederma, combining ceramide 2, cement of the stratum corneum, and Pal-Val-Gly-Val-Ala-Pro-Gly, a palmitoylated matrikine which smoothes wrinkles and repairs the skin barrier.
An anti-wrinkle/anti-aging ingredient based on pro-collagen peptide(s) such as: MATRIXYL™, MATRIXYL 3000™, MATRIXYL synthe′6™ and/or MATRIXYL Morphomics™ marketed by Sederma.

2—Mild Aqueous Serum Form

TABLE 20

| Ingredients | INCI name | Role | % |
|---|---|---|---|
| Part A: | | | |
| H₂O | / | / | qsp 100 |
| Potassium sorbate | Potassium Sorbate | Preservative | 0.10 |
| Part B: | | | |
| Glycerin | Glycerin | Humectant | 5.00 |
| Phenoxyethanol | Phenoxyethanol | Preservative | 0.80 |
| Part C: | | | |
| Cromollient ™ SCE | Di-PPG-2 Myreth-10 Adipate | Emollient | 1.20 |
| VisiaOptima ™ SE | Sodium Polyacrylate (and) | Rheology modifier | 1.00 |
| | Ethylhexyl Cocoate (and) | and emulsion stabiliser | |
| | PPG-3 Benzyl Ether Myrisate | | |
| | (and) Polysorbate 20 | | |
| Part D: | | | |
| H₂O | / | / | 0.80 |
| NaOH 30% | Sodium Hydroxide | pH adjuster | 0.08 |
| Part C: | | | |
| Ingredient according to | / | Active | 3.00 |
| the invention | | | |

Example(s) of Additional Active Ingredient(s):
an anti-aging active ingredient such as:
SENESTEM™, marketed by Sederma, comprising plant cells obtained by in-vitro cell culture of Plantago lanceolata, which in particular improves skin viscoelastic properties and lightens senescence age spots.
un antioxidant ingredient such as:
MAJESTEM™, marketed by Sederma, based on leontopodium alpinum plant cells obtained by in-vitro cell culture titrated in leontopodic acid; neutralizing oxidative stress (pollution and UV radiation) and restoring skin tension.

3—Gel Form

TABLE 21

| Ingredients | INCI name | Role | % |
|---|---|---|---|
| Part A: | | | |
| H₂O | / | / | qsp 100 |
| Carbomer | / | Rheology | 0.40 |
| | | modifier | |
| Part B: | | | |
| Glycerin | Glycerin | Humectant | 7.00 |
| Phenoxyethanol | Phenoxyethanol | Preservative | 0.80 |
| Part C: | | | |
| H₂O | / | / | 3.00 |
| NaOH 30% | Sodium Hydroxide | pH adjuster | 0.30 |
| Part D: | | | |
| Tween ™ 20 | Polysorbate 20 | Emulsifier | 0.50 |
| Cromollient ™ SCE | Di-PPG-2 Myreth-10 Adipate | Emollient | 1.00 |
| Covi-ox ™ | Tocopherol (and) Helianthus | Antioxidant | 0.40 |
| | Annuus (Sunflower) Seed Oil | | |
| Part E: | | | |
| Ingredient according to | / | Active | 3.00 |
| the invention | | | |

Example(s) of Additional Active Ingredient(s):
an “antipollution” ingredient such as:
CITYSTEM™, marketed by Sederma, based on plant cells obtained in vitro from Marrubium vulgare with a high concentration of Forsythoside B; used against pollution attacks: makes the skin soft and smooth, refines the skin texture, reduces the visibility of blackheads, leaving the skin radiant and purified.
a calming ingredient for sensitive skin such as:
PACIFEEL™, marketed by Sederma, comprising an extract of Mirabilis Jalapa.
A moisturizing ingredient such as:
AQUALANCE™, marketed by Sederma, an osmoprotective moisturizer composed of homarin and erythritol.

4—Gel Form to Realize a Spray Mask

TABLE 22

| Ingredients | INCI name | Role | % |
|---|---|---|---|
| Part A: | | | |
| H₂O | / | / | qsp 100 |
| Hydrotriticum PVP PE ™ | Aqua (and) Hydrolyzed Wheat | Film-forming | 3.00 |
| | Protein/PCP Crosspolymer | agent | |
| Volarest ™ FL | Acrylates/Beheneth-25 | Rheology | 2.30 |
| | Methacrylate Copolymer | modifier | |
| Potassium sorbate | Potassium Sorbate | Preservative | |
| Part B: | | | |
| Glycerin | Glycerin | Humectant | 5.00 |
| Phenoxyethanol | Phenoxyethanol | Preservative | 0.80 |
| Part C: | | | |
| Crovol ™ A70 | PEG-60 Almond Glycerides | Emollient | 1.00 |
| Ethanol | Ethanol | Solvent | 5.00 |
| Covi-ox ™ | Tocopherol (and) Helianthus | Antioxidant | 0.20 |
| | Annuus (Sunflower) Seed Oil | | |
| Part D: | | | |
| H₂O | / | / | 2.50 |
| NaOH 30% | Sodium Hydroxide | pH adjuster | 0.25 |
| Part E: | | | |
| Ingredient according to | | Active | 3.00 |
| the invention | | | |

Example(s) of additional active ingredient(s):
an ingredient acting on the radiance of the complexion such as:
EVERMAT™, marketed by Sederma, comprising a combination of an extract of Enantia chlorantha rich in protobberberins and oleanolic acid; decreases the pore size and skin shine; refines the texture of acne-prone skin.
an ingredient with revitalizing properties such as:
Fruitliquid™ Kumquat™, marketed by Crodarom.

5—Cream Form, for a Make-Up Base

TABLE 23

| Ingredients | INCI name | Role | % |
|---|---|---|---|
| Part A: | | | |
| H₂O | / | / | qsp 100 |
| Volarest ™ FL | Acrylates/Beheneth-25 Methacrylate | Rheology | 0.90 |
| | Copolymer | modifier | |
| Part B: | | | |
| Arlacel ™ 2121 | Sorbitan Stearate (and) Sucrose | Emulsifier | 4.50 |
| | Cocoate) | | |
| Partie C : | | | |
| Pentylène glycol | Pentylene Glycol | Humectant | 5.00 |
| Phénoxyéthanol | Phenoxyethanol | Preservative | 0.80 |
| Partie D : | | | |
| Crodamol ™ SSA | Decyl Isostearate (and) Isostearyl | Emollient | 2.00 |
| | Isostearate | | |
| Crodamol ™ TN | Isotridecyl Isononanoate | Emollient | 2.00 |
| Crodamol ™ AB | C12-C15 Alkyl Benzoate | Emollient | 1.50 |
| Crodamol ™ GTEH | Triethylhexanoin | Emollient | 3.00 |
| Covi-ox ™ | Tocopherol (and) Helianthus Annuus | Antioxidant | 0.10 |
| | (Sunflower) Seed Oil | | |
| Part D: | | | |
| Potassium sorbate | Potassium Sorbate | Preservative | 0.10 |
| Part E: | | | |
| H₂O | / | / | 2.50 |
| NaOH 30% | Sodium Hydroxide | pH adjuster | 0.25 |
| Part E: | | | |
| Ingredient according | | Active | 3.00 |
| to the invention | | | |

Example(s) of additional active ingredient(s):
An ingredient for treating under eye bags and dak circles such as: HALOXYL™, marketed by Sederma, a combination of two matrikines, the Pal-GHK and the Pal-GQPR with N-hydroxysuccinimide and a flavonoid, the chrysin.
EYELISS™, marketed by Sederma, combining three components: hesperidin methyl chalcone, the dipeptide Valyl-Tryptophan (VW) and the lipopeptide Pal-GQPR.
PRODIZIA™, marketed by Sederma, comprising an extract of Albizia julibrissin, which promotes the visible reduction of signs of fatigue: dark circles, under eye bags, dull complexion and drawn features by repairing and protecting the skin from damage caused by glycation.
An anti-wrinkle/anti-aging ingredient based on pro-collagen peptide(s) such as:
MATRIXYL™, MATRIXYL 3000™, MATRIXYL synthe′6™ and/or MATRIXYL Morphomics™ marketed by Sederma.

6—Cream Form, for Example for Treating an Acne Prone Skin (Cream Used for the In Vivo Efficacy Tests Given Below)

TABLE 24

| Ingredient | INCI name | Role | % |
|---|---|---|---|
| Part A | | | |
| demineralized water | Aqua | / | qsp 100 |
| Carbopol Ultrez 10 ™ | Carbomer | Thickener/Gelling agent | 0.45 |
| Part B | | | |
| Glycerin | Glycerin | Humectant | 2.00 |
| Octanediol | Caprylyl Glycol | Humectant/Emollient | 0.50 |
| Part C | | | |
| Phenoxyethanol | Phenoxyethanol | Preservative | qs |
| Part D | | | |
| Squalane | Squalane | Emollient | 3.00 |
| Crodamol AB ™ | C12-15 Alkyl Benzoate | Emollient | 2.00 |
| Pemulen TR2 ™ | Acrylates/C10-30 Alkyl | Emulsifier | 0.20 |
| | Acrylate Crosspolymer | | |
| Part E | | | |
| Potassium sorbate | Potassium Sorbate | Preservative | qs |
| Part F | | | |
| Demineralized water | Aqua | / | 5.00 |
| NaOH 30% | Sodium Hydroxide | pH adjuster | 0.50 |
| Part G | | | |
| Ingredient according to the | | Active | 1.00 |
| invention | | | |
| Part H | | | |
| Tween 20 | Polysorbate 20 | Emusifier | 1.00 |
| Perfume | Fragrance | / | 0.10 |

Example(s) of additional active ingredient(s):
An ingredient for treating more specifically epidermis, such as:
CRYSTALIDE™, marketed by Sederma, comprising the Pal-KTFK peptide in a solvated form.
An ingredient for resynchronising the skin against digital pollution damage such as:
SYNCHROLIFE™, marketed by Sederma, comprising chrysin, rosmarinic acid and the Pal-GQPR peptide.
An ingredient for lightening skin such as:
LUMISKIN™, marketed by Sederma, comprising a noraporphine derivative.
An ingredient for treating hormonal facial skin sagging such as:
FEMINAGE™, marketed by Sederma, comprising a plant extract of the Engelhardtia chrysolepsis.

E—In-vivo efficacy tests

The efficacy evaluation of the peptide(s) according to the invention was carried out at 28 and 56 days, on a total of 48 volunteers, in two independent studies against placebo. The effect on various imperfections of acne-prone skins: atrophic scars (in depth residual scars), residual-coloured marks (red and/or brown traces), inflammatory lesions (created or left by acneic phenomenons, such as pimples or pustules), roughness, redness overall skin uniformity, were evaluated thanks to these studies.

The objective of the first study was to quantify the cutaneous imperfections using a multiparameter 2D-3D camera and an expert evaluation on photos.

The second study associated an evaluation by a dermatologist, an image analysis on standardized photos and the perceived effect by the volunteers. The synopsis of these studies is shown on the below:

TABLE 25

| T0 | T 28 days | T 56 days |
|---|---|---|
| Expert evaluation on | | Expert evaluation on |
| photos | | photos |
| 2D-3D camera evaluation | | 2D-3D camera evaluation |
| Dermatologist evaluation | Dermatologist evaluation | Dermatologist evaluation |
| Perceived effect | Perceived effect | |
| Image analysis | | Image analysis |

Used Cream:

The cream formula 6 (Table 24) given in the above Galenical part, the placebo cream comprising the same formula without the active ingredient according to the invention (comprising 1200 ppm of Pal-KTSKS-OH, 2HCl).

1) First Study:

Protocol:

Panel description: the study was carried out on a panel of 18 volunteers (17 women and 1 man) of average age 32 years (20-58 years) and of phototype II to IV. They had an oily skin with atrophic acne scars and almost all acne lesions. None of them were undergoing specific treatment for acne-prone skin.

Study type, duration, and applications: single-blind study, the volunteers using twice a day on the half of the face the cream according to the invention or its placebo; the applications lasted two months with an intermediate time of one month.

Statistics: for the imperfection quantification, statistical studies were performed with a Student t test or if necessary with a nonparametric Wilcoxon test. Bilateral tests were carried out on paired series.

For the expert assessment: a Khi2 test was used to compare response frequencies.

Methods and Results:

Expert Evaluation on Photos:

A photographic bench was used to acquire the photos to ensure perfect repositioning at each stage of the study. For each volunteer, profile photos were taken in a parallel polarized mode, therefore with shine, to obtain a clearer view of the relief in depth (acne-related scars) while visualizing the other imperfections (acne lesions in height and residual marks).

The obtained photos were assessed by a panel of six expert judges. For each volunteer, the experts viewed the photos before and after treatment and gave their opinion on the following statement: the product has reduced the imperfections (acne-related scars, residual marks and lesions). The responses reflect 43% favorable opinions in favor of the cream according to the invention, which is significantly higher than the 21% favorable opinions for the placebo.

Evaluation Using a Multiparametric Camera:

A portable LED camera illuminating in the visible at seven wavelengths was used. In addition, the skin surface was illuminated from different angles, allowing the image to be reconstructed in three dimensions. Polarizing filters were used to remove the shine and the use of a ALS (Ambient Light Substraction) algorithm ensured complete independence from external lighting. Acquisition without skin contact provides a measurement field of 5.6×5.6 cm with lateral (x-y) resolution of 0.1 mm and vertical (z) resolution of 0.01 mm.

This system allows a multi analysis of:
the L* a* b* color parameter (L* value indicates lightness, and a* and b* are chromaticity coordinates);
The chromophore concentrations such as hemoglobin and melanin; and
Various topographies such as texture, pore volumes, scars or lesions

Results on Skin Smoothing:

Atrophic scars were particularly targeted in the analysis area.

The reliefs in height (inflammatory lesions and pimples), in depth (atrophic scars) as well as the global roughness, to follow a smoothing of the skin, were studied.

TABLE 26
Variation in the volume of lesions and scars (N = 18 volunteers)

| Volume of the lesions (in mm³) | | Volume of the scars (in mm³) | |
|---|---|---|---|
| Invention | Placebo | Invention | Placebo |

| | T0 | T56 | T0 | T56 | T0 | T56 | T0 | T56 |
|---|---|---|---|---|---|---|---|---|
| Mean | 2.12 | 1.29 | 3.07 | 2.92 | 3.86 | 2.97 | 4.37 | 4.19 |
| Standard deviation | 1.78 | 1.25 | 2.72 | 2.49 | 2.69 | 2.34 | 3.60 | 3.51 |
| % variation vs. T0 | | −39.2% | | −4.9% | | −23% | | −4.1% |
| Significativity | | p < 0.01 | | nsd | | p < 0.01 | | nsd |
| Maximum | | −96% | | | | −86% | | |
| Responders | | 94% | | | | 100% | | |
| Significativity vs. | | <0.05 | | | | <0.01 | | |
| Placebo | | | | | | | | |

TABLE 27
Variation of the global roughness (N = 18 volunteers)

| | Global roughness (in μm) | | | |
|---|---|---|---|---|
| | Invention | | Placebo | |
| | T0 | T56 | T0 | T56 |
| Mean | 11.42 | 10.50 | 11.84 | 11.65 |
| Standard deviation | 2.55 | 2.78 | 3.45 | 3.34 |
| % variation vs. T0 | | −8.1% | | −1.6% |
| Significativity | | p < 0.01 | | nsd |
| Maximum | | −25% | | |
| Responders | | 100% | | |

| | |
|---|---|
| Significativity vs. Placebo | p < 0.01 |

A significant decrease in lesions and scars is observed with the application of the cream according to the invention while the placebo has no significant effect. Skin smoothing is thus clearly demonstrated thanks to the invention cream, which is confirmed with the overall roughness parameter which shows a reduction of 8.1%.

Results on Redness:

Two parameters were highlighted: maximum redness, which is the most effective parameter to see a reduction in severe redness of the lesion-type, and the heterogeneity of redness which quantifies the extent of redness in the analyzed surface.

TABLE 28
Variation of redness (N = 18 volunteers)

| Concentration/ | Maximum redness | | | | Redness heterogeneity | | | |
|---|---|---|---|---|---|---|---|---|
| Heterogeneity | Invention | | Placebo | | Invention | | Placebo | |
| (in arbitrary units) | T0 | T56 | T0 | T56 | T0 | T56 | T0 | T56 |
| Mean | 44.96 | 39.48 | 43.15 | 40.93 | 2.87 | 2.52 | 2.82 | 2.67 |
| Standard deviation | 12.62 | 12.95 | 8.86 | 9.09 | 1.32 | 1.13 | 1.10 | 0.96 |
| % Variation vs. T0 | | −12.2% | | −5.2% | | −12.0% | | −5.4% |
| Significativity | | p < 0.01 | | nds | | p < 0.01 | | nds |
| Maximum | | −36% | | | | −31% | | |
| Responders | | 89% | | | | 72% | | |
| Significativity vs. | | p < 0.05 | | | | p < 0.05 | | |
| Placebo | | | | | | | | |

The association of a decrease of the maximum redness with a reduction in heterogeneity makes residual marks or inflammatory lesions less visible and the complexion more uniform.

2) Second Study

Protocol:

Panel description: the study was carried out on a panel of 30 volunteers (7 men and 23 women) of average age 24 years (18-39 years), of phototype III and with oily or mixte skin. They all had an oily skin with inflammatory lesions, residual red or brown traces, and most (26) had atrophic acne scars grade 2-4 on the Goodman scale. None of them were undergoing specific treatment for acne-prone skin.

Type of Study, Duration, and Applications:

For this single-blind study, the volunteers used the cream according to the invention or its placebo twice a day on the half of the face. The applications lasted 2 months with an intermediate time of 1 month.

Statistics:

For the clinical evaluation, a linear mixed-effects model was used which is suitable for unbalanced designs (missing datas as 3 panelists were missing at T2months). The analysis was therefore performed on the set of subjects, whether or not they have missing data. This part makes it possible to say whether there is globally a product effect, a time effect, and a product x time interaction. Subsequently, in a more conventional fashion, pairwise comparisons were made using a post-hoc Tukey test.

For the image analysis and the effect perceived by the volunteers, a Khi2 test was used to compare the response frequencies.

Methods and Results:

Clinical Evaluation

It was carried out at T0, T28 and T56 by the same dermatologist for each half-face while respecting a standardized position and lighting of the volunteer.

A count of the inflammatory lesions (pimples and pustules) was carried out according to the method of Lucky et al. Each type of imperfection was counted on the following areas: forehead, cheeks, and chin (right and left side excluding the nasal pyramid, vermilion border, chin crease and scalp edge). Residual marks (red and brown) were counted similarly.

Regarding atrophic scars, the dermatologist assessed their severity on a scale of 0 (no scars) to 9 (scars of severe intensity in relation to their appearance and number).

TABLE 29
Variation of inflammatory lesions and residual marks
(N = 30 volunteers at T28, 27 volunteers at T56)

| | Inflammatory lesions | | | | | |
|---|---|---|---|---|---|---|
| Numbers | Invention | | | Placebo | | |
| of lesions | T0 | T28 | T56 | T0 | T28 | T56 |
| Mean | 10.5 | 6.1 | 4.7 | 10.6 | 8.5 | 7 |
| Standard deviation | 6.2 | 4.3 | 4.3 | 5.9 | 4.4 | 5.2 |
| % Variation vs. T0 | | −42% | −55% | | −20% | −34% |
| Significativity | | p < 0.01 | p < 0.01 | | p = 0.06 | p < 0.01 |
| Maximum; | | −100%; | −100%; | | | |
| Responders | | 90% | 93% | | | |
| Significativity | | p < 0.05 | p < 0.05 | | | |
| vs. Placebo | | | | | | |

TABLE 30
Variation of the residual marks (N = 30 volunteers at T28, 27 volunteers at T56)

| | Residual marks | | | | | |
|---|---|---|---|---|---|---|
| Numbers | Invention | | | Placebo | | |
| of marks | T0 | T28 | T56 | T0 | T28 | T56 |
| Mean | 15.7 | 12.3 | 11.6 | 15.4 | 14.6 | 13.9 |
| Standard deviation | 8.6 | 6.8 | 7.1 | 7.8 | 6.8 | 5.7 |
| % Variation vs. T0 | | −22% | −26% | | −5% | −10% |
| Significativity | | p<0.01 | p<0.01 | | nds | nds |
| Maximum; Responders | | −75%; | −87%; | | | |
| | | 87% | 89% | | | |
| Significativity | | p = 0.08 | p = 0.12 | | | |
| vs. Placebo | | | | | | |

TABLE 31
Variation of atrophic scars (N = 30 volunteers at T28, 27 volunteers at T56)

| | Severity of the atrophic scars | | | | | |
|---|---|---|---|---|---|---|
| Score of the atrophic | Invention | | | Placebo | | |
| scars (0 to 9) | T0 | T28 | T56 | T0 | T28 | T56 |
| Mean | 4.6 | 4.2 | 3.7 | 4.4 | 4.3 | 4.1 |
| Standard deviation | 1.5 | 1.6 | 1.4 | 1.6 | 1.6 | 1.6 |
| % variation vs. T0 | | −9% | −20% | | −2% | −7% |
| Significativity | | p < 0.05 | p < 0.01 | | nds | p = 0.09 |
| Maximum; Responders | | −33%; | −40%; | | | |
| | | 33% | 67% | | | |
| Significativity vs. Placebo | | nds | p < 0.05 | | | |

These results show that the application of the cream of the invention causes a significant reduction in lesions, residual marks and scars. These effects, present as early as 28 days, are accentuated after 56 days of treatment and are significantly greater and globally significant compared to those provided by the placebo.

Image Analysis on Standardized Photos

The standardized photos were taken with a HeadScan Dynamics System II (OrionTechnoLab, France) equipped with a professional camera and a high resolution (24 Mpx) sensor. Photos in cross-polarized and scattered light were obtained with an automatic system for positioning different filters. Subject repositioning during the different study stages was ensured by a restraint device and a laser line projected on the corners of the mouth.

The image analysis used in the study tracked the number of inflammatory lesions and their intensity. The results below show the % of volunteers who saw improvement in their condition.

TABLE 32
Improvement in the number and intensity of the lesions after
56 days of treatment (in % of volunteers; N = 27 volunteers)

| | | | | Numbers and intensity of | |
|---|---|---|---|---|---|
| Numbers of lesions | | Intensity of the lesions | | the lesions | |
| Invention | Placebo | Invention | Placebo | Invention | Placebo |
| 70.4%* | 55.6% | 74.1%*^{$} | 33.3% | 51.9%^{$} | 18.5% |

*significant variation vs. T0 with p < 0.05,
^{$}significant variation vs. Placebo with p < 0.05

Among the volunteers who applied the cream according to the invention, 70.4% had a decrease in the number of lesions, 74.1% a decrease in the intensity of the lesions and 51.9% the combination of the two, against only respectively 55.6%, 33.3% and 18.5% for the placebo.

Effect perceived by volunteers: perceived effect was assessed using a self-report questionnaire after 28 days of treatment.

TABLE 33
For each statement, the volunteers had
to choose from the following answers:

| | |
|---|---|
| Totaly agree | Grouped into « favorable opinion » |
| Agree | |
| Disagree | Grouped into « defavorable opinion » |
| Totaly disagree | |

TABLE 34
Perceived efficacy after 28 days of treatment (in % of favorable
opinions; N = 30 volunteers)

| Less scars | | Attenuated scars | | Less rednesses | | Less visible pores | |
|---|---|---|---|---|---|---|---|
| Invention | Placebo | Invention | Placebo | Invention | Placebo | Invention | Placebo |
| 92%^{$} | 38% | 92%^{$} | 62% | 100%^{$} | 83% | 97%^{$} | 57% |

| Less imperfections | | More uniform skin | | Less rednesses | |
|---|---|---|---|---|---|
| Invention | Placebo | Invention | Placebo | Invention | Placebo |
| 83%^{$} | 50% | 97%^{$} | 73% | 83%^{$} | 37% |

^{$}Significant variation vs. placebo with p < 0.05

The results show an efficacy of the cream according to the invention that is systematically greater than the placebo and shows a perceived effect in terms of scars (92%), residual marks and imperfections (83%), pores and skin uniformity (97%) as well as rednesses (100%).

All these in vivo results show that the peptide(s) according to the invention can embellish the skin by acting on all type of cutaneous imperfections in relief and in colours, in particular imperfections that are due to residual scars and marks, more particularly due to acne episodes.

1.-35. (canceled)

36. A method of treating keratinic materials of skin and its integuments, comprising applying to an epidermis, scalp, hair and nails in need thereof of at least one peptide of the following general Formula 1 or a composition comprising said at least one peptide and a physiologically acceptable medium:
X—(Xaa)ₙK*TSK*X′aa-(Xaa)ₘ-Z (1)
wherein in general Formula 1:
K* is chosen from lysine, hydroxylysine, ornithine, diaminobutyric acid or diaminopropionic acid or their formyl, acetyl, trifluoroacetyl, methanesulfonyl or succinyl derivatives, the two K* possibly being identical or different;
(Xaa)ₙ and (Xaa)ₘ correspond independently of one another to a sequence of n or m amino acids Xaa chosen independently of one another from G, A, P, V, L, I and F, with n and m being integers which may be equal or different between 0 and 5;
X′aa is chosen from threonine and serine;
at the N-terminal end X is chosen from H, —CO—R¹, —SO₂-R¹ or a biotinoyl group;
at the C-terminal end Z is chosen from OH, OR¹, NH₂, NHR¹ or NR¹R²; and
R¹ and R² are, independently of one another, chosen from an alkyl, aryl, aralkyl, alkylaryl, alkoxy, saccharide and aryloxy group, which may be linear, branched, cyclic, polycyclic, unsaturated, hydroxylated, carbonylated, phosphorylated and/or sulfurized, said group having from 1 to 24 carbon atoms and possibly having in its backbone one or more O, S and/or N heteroatoms.

37. The method according to claim 36, wherein the treatment is a cosmetic treatment for:
treating the epidermal stratum corneum for protecting the epidermis and scalp from external aggressions liable to cause damage, such as microorganisms, radiation and molecules;
smoothing epidermis relief;
uniformizing skin epidermis;
treating skin pores;
treating oily and/or acne-prone skins; and/or
preserving the balance of the skin microbiota.

38. The method according to claim 37, for preventing cutaneous redness, irritation, and tightness, and/or or premature aging of the epiderm, for preventing the scalp against the appearance of dandruff by inhibiting the development of the yeasts responsible for a dandruff condition of the Malassezia genus and/or for treating epidermis dehydration.

39. The method according to claim 37, for smoothing acne atrophic traces.

40. The method according to claim 37, for treating coloured marks.

41. The method according to claim 40, for treating residual acne lesion-coloured marks.

42. The method according to claim 37, wherein the appearance of acne is prevented, by inhibiting the multiplication of the P. acnes bacteria responsible for acne.

43. The method according to claim 36, for an antimicrobial and/or anti-inflammatory treatment.

44. The method according to claim 43, for inhibiting the growth of the Propionibacterium acnes bacteria responsible for acne and/or yeasts of the Malassezia genus responsible for a dandruff state.

45. The method according to claim 43 for an anti-inflammatory composition suitable for soothing sensitive and irritated skin.

46. The method according to claim 43 for treating acne, psoriasis, dermatitis and/or eczema.

47. The method according to claim 36, wherein the at least one peptide is used in a vectorized form, being bound, incorporated or adsorbed on/to macro-, micro- or nanoparticles. such as capsules, spheres, liposomes, oleosomes, chylomicrons, sponges, in the form of micro- or nano-emulsions, or adsorbed for example on powdery organic polymers, talcs, bentonites, spores or exins and other inorganic or organic supports.

48. The method according to claim 36, wherein K* is a lysine or an ornithine.

49. The method according to claim 36, wherein X′aa is serine.

50. The method according to claim 36, wherein n and m are independently of each other 0 or 1 or 2.

51. The method according to claim 36, wherein the peptide is modified in the N-terminal position and/or in the C-terminal position.

52. The method according to claim 36, wherein R¹ and/or R² is an alkyl chain of 1 to 24 carbon atoms.

53. The method according to claim 52, wherein R¹ and/or R² is an alkyl chain of 3 to 24 carbon atoms.

54. The method according to claim 36 wherein X is an acyl group CO—R¹ and Z is chosen from OH, OMe, OEt and NH₂.

55. The method according to claim 36 wherein said peptide is Pal-KTSKS (SEQ ID No 5).
